(19) United States
(12) Patent Application Publication
(10) Pub. No.: US 2026/0241016 A1
(43) Pub. Date: Aug. 20, 2026

(54) PEPTIDE-DEXAMETHASONE CONJUGATE, PREPARATION METHOD THEREOF, AND APPLICATION METHOD THEREFOR

(71) Applicant: Shandong University, Jinan City (CN)

(72) Inventors: Fengshan WANG, Jinan City (CN); Xinke ZHANG, Jinan City (CN); Xiaohan XU, Jinan City (CN)

(73) Assignee: Shandong University, Jinan City (CN)

(21) Appl. No.: 19/399,644

(22) Filed: Nov. 25, 2025

(30) Foreign Application Priority Data
Nov. 26, 2024 (CN) 202411696979.0

(51) Int. Cl. A61K 47/64; A61K 47/54; A61P 27/02

(52) U.S. Cl. CPC A61K 47/64; A61K 47/545; A61P 27/02

(57) ABSTRACT

The present disclosure belongs to the field of biomedicine technology and relates to a peptide-dexamethasone conjugate, preparation method therefor, and application method thereof. The peptide-dixamethasone conjugate has the chemical structure shown in Formula I;

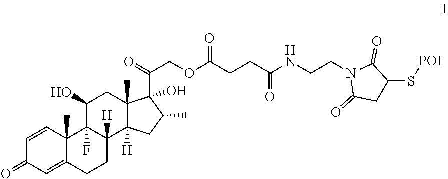

where POI is a peptide, the peptide includes a short peptide and cysteine residues. The short peptide includes one or more of RGD, TAT, and AT7, and the POI is connected to the maleimide group via thioether bonds through cysteine residues. The peptide-dexamethasone conjugate provided by the present disclosure has targeting, penetrating, anti-inflammatory, and anti-neovascular activity, and can be better used for targeted therapy of neovascular eye diseases.

### CROSS-REFERENCE TO RELATED APPLICATION

This application claims priority to Chinese Patent Application No. 202411696979.0, filed on Nov. 26, 2024, which is hereby incorporated by reference in its entirety.

### REFERENCE TO SEQUENCE LISTING XML

The present application contains a Sequence Listing XML submitted electronically herewith as an XML file named 2024806650.xml, created on Nov. 25, 2024, and having a size of 5,126 bytes. The information contained in the Sequence Listing XML is incorporated herein by reference in its entirety.

### TECHNICAL FIELD

The present disclosure belongs to the field of biomedicine technology, and relates to peptide-dexamethasone conjugate, preparation method thereof, and application method therefor.

### BACKGROUND ART

The information disclosed in the background art is intended solely to enhance the understanding of the overall background of the present disclosure and should not necessarily be regarded as acknowledging or in any way implying that this information constitutes prior art that has become generally known to those skilled in the art.

Neovascular eye disease is a type of eye disease defined by excessive neovascular formation and abnormal vascular remodeling in the eye. Vascular endothelial growth factor (VEGF) is widely recognized as one of the important therapeutic targets for neovascular eye diseases. Almost all anti-VEGF biologics used in tumor treatment have undergone clinical trials for ocular diseases, such as bevacizumab, ranibizumab, and aflibercept. However, anti-VEGF treatment also has some limitations. Firstly, its duration of action is relatively short, and there is insufficient response, with a significant portion of patients experiencing poor vision recovery after treatment. Additionally, intravitreal injection of anti-VEGF drugs often accompanies inflammation-related adverse reactions. Currently, it has been observed that the pathogenesis of ocular neovascular-related diseases is multifactorial, in addition to VEGF, other factors may also be involved. Studies have shown that inflammation plays an important role in the pathogenesis of neovascular eye diseases. As one of the highly effective anti-inflammatory drugs, glucocorticoids have broad application prospects in treating inflammation-related diseases. Glucocorticoid intravitreal implants have served as an alternative therapy in the treatment of fundus diseases alongside anti-VEGF therapy. Clinical studies have found that, compared to single anti-VEGF therapy, patients with refractory diabetic macular edema (DME) showed more significant improvements in macular fovea thickness and vision after receiving combined treatment with anti-VEGF drugs and glucocorticoids. Therefore, there is a need to provide a drug for treating neovascular diseases that is well-tolerated and based on multiple factors.

Peptide-drug conjugates (PDC) have received increasing attention as a type of targeted drug that can be designed for multiple targets. PDCs are a class of drugs formed by the covalent binding of peptides with other bioactive molecules (such as drugs, antibodies, fluorescent dyes, etc.), with the advantage of utilizing the targeting ability of peptides to deliver drugs to specific cellular or tissue targets, thereby enhancing drug efficacy and reducing side effects. The inventors of the present disclosure designed an anti-angiogenic dual-target penetrating peptide TAT-AT7 in previous research, which was lipid-modified and made into a hydrogel formulation for ocular administration. TAT-AT7 demonstrated excellent ability to penetrate the ocular barrier and inhibit retinal neovascularization. However, further research by the inventors found that although the strong penetrating ability of the peptide TAT-AT7 allows it to penetrate the ocular barrier, it does not effectively concentrate its action on the lesion site. Therefore, it is necessary for further improving the drugs.

### SUMMARY

In order to address the shortcomings of the prior art, the objective of the present disclosure is to provide a peptide-dexamethasone conjugate, preparation method thereof, and application method therefor, the peptide-dexamethasone conjugate possesses targeting, penetrating, anti-inflammatory, and anti-neovascular activities, and can be better used for targeted treatment of neovascular eye diseases.

To achieve the above objectives, the technical solution of the present disclosure is as follows.

In the first aspect, the peptide-dexamethasone conjugate has the chemical structure shown in formula I;

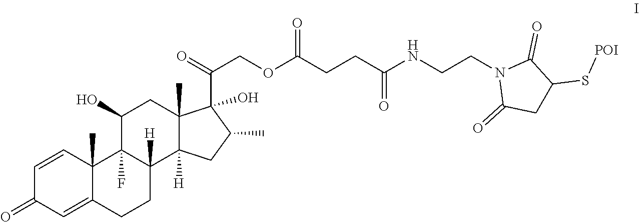

where POI is a peptide, the peptide includes short peptides and cysteine residues, the short peptides include: one or more of RGD (amino acid residue sequence is GRGDS, as shown in SEQ ID NO. 1), TAT (amino acid residue sequence is RKKRRQRRR, as shown in SEQ ID NO. 2), and AT7 (amino acid residue sequence is ATWLPPR, as shown in SEQ ID NO. 3), the POI is connected by cysteine with maleimide groups via thioether bonds.

TAT is a class of representative cell-penetrating peptides, due to its strong penetrating ability, it shows unique advantages in transmembrane transduction and penetrating physiological barriers such as the blood-brain barrier (BBB), blood-retinal barrier (BRB), etc. It has been proven to carry drug molecules and has shown great advantages in the treatment of gliomas, central nervous system diseases, and ocular neovascular diseases. The peptide AT7 is a vascular-targeting peptide screened from a phage library, the amino acid residue sequence is ATWLPPR, which can produce a dual-targeting effect by simultaneously binding to vascular epidermal growth factor receptor-2 (VEGFR-2) and neuropiliary protein-1 (NRP-1), inhibiting the angiogenic effect of VEGF, and has higher selectivity for the ocular neovascular system. The targeted penetrating peptide TAT-AT7 constructed by coupling peptide TAT and peptide AT7 has significant anti-angiogenic activity and blood-brain barrier permeability.

Integrins area type of cell adhesion protein, which play an important role in promoting the adhesion of endothelial cells to the extracellular matrix. Integrins are composed of different subtypes, among which integrin αᵥβ₃ has the strongest regulatory effect on angiogenesis and is highly expressed on the surface of endothelial cells in neovascularization. Ruoslahti et al. first discovered that the peptide RGD is the binding sequence of the extracellular matrix and integrin αᵥβ₃, which has strong selectivity and affinity for integrin αᵥβ₃.

Based on this, the present disclosure connects the anti-angiogenic penetrating peptide TAT-AT7 and/or integrin-targeting peptide ROD and small molecule anti-inflammatory drug dexamethasone through ester bonds, amide bonds, and maleimide-thioether bonds, constructing a novel peptide-dexamethasone conjugate that has targeting, penetrating, anti-inflammatory, and anti-neovascular activity.

In some embodiments, the short peptide is RGD, or a peptide formed by TAT and AT7, or a peptide formed by ROD, TAT, and AT7.

In some embodiments, the short peptide is a peptide formed by RGD, TAT, and AT7. Studies have shown that when the short peptide is a peptide formed by RGD. TAT, and AT7, the effect of the conjugate is better.

Specifically. TAT and AT7 form the TAT-AT7 peptide, and the cysteine residue is located between TAT and AT7.

Specifically, the amino acid residue sequence of POI is: GRGDSRKKRRQRRRCATWLPPR, as shown in SEQ ID NO. 4.

In the second aspect, a preparation method of the above-mentioned peptide-dexamethasone (DEX) conjugate is provided, which esterifies dexamethasone with succinic anhydride to obtain DEX-SA, then conducts an amidation condensation reaction of DEX-SA with N-(2-aminoethyl) maleimide to obtain DEX-MAL, and then performs an addition reaction of DEX-MAL with POI-SH (thiolated peptide) to obtain the above-mentioned peptide-dexamethasone (DEX) conjugate:
where the chemical structure of DEX-SA is expressed as:

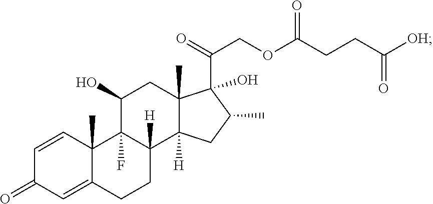

the chemical structure of DEX-MAL is expressed as:

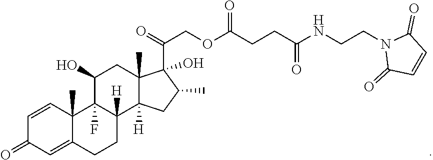

In some embodiments, dexamethasone is reacted with succinic anhydride and 4-dimethylaminopyridine (DMAP) at room temperature to obtain DEX-SA. Specifically, the solvent is acetone. Specifically, the mass ratio of dexamethasone to succinic anhydride and 4-dimethylaminopyridine is 1.3~1.7:0.70~0.80:0.15~0.20. The room temperature referred to in the present disclosure means the indoor environmental temperature, generally 15~30° C.

In some embodiments, 2-(7-azabenzotriazole)-N,N,N′,N′-tetramethylurea hexafluorophosphate (HATU), 1-hydroxy benzotriazole (HOBT), and N,N-diisopropylethylamine (DIPEA) are used to activate the carboxylic acid in DEX-SA, and then N-(2-aminoethyl) maleimide is added to perform an amidation condensation reaction to obtain DEX-MAL. Specifically, the reaction temperature is room temperature.

In some embodiments, the condition for the addition reaction of DEX-MAL with POI-SH is that the reaction is performed at room temperature for 1.5~3 hours.

In the third aspect, a pharmaceutical composition is provided, which includes the above-mentioned peptide-dexamethasone conjugate, as well as pharmaceutical excipients.

The pharmaceutical excipients described in the present disclosure include pharmaceutical carriers and/or excipients. The pharmaceutical carriers include but are not limited to serum proteins, water, physiological saline, phosphate buffer solutions, etc. The excipients include but are not limited to antioxidants, buffers, pH adjusters, fillers, emulsifiers, co-emulsifiers, binders, preservatives, flavors, etc.

In the fourth aspect, a pharmaceutical formulation is provided, which includes nanomicelles, which are formed by the above-mentioned peptide-dexamethasone conjugate.

Dexamethasone end has a hydrophobic nature, while the peptide end has a hydrophilic nature, thus the conjugate can self-assemble into nanomicelles in an aqueous solution.

In some embodiments, the conjugate is prepared into nanomicelles using a vortex ultrasound method.

In some embodiments, the particle size of the nanomicelles is 100-200 nm, and the Zeta potential is positively charged.

In the fifth aspect, an application method of the above-mentioned peptide-dexamethasone conjugate, pharmaceutical composition, or pharmaceutical formulation in the preparation of a drug for treating neovascular eye diseases is provided.

In vitro, in aqueous humor, monitored by HPLC, most of the peptide-dexamethasone conjugate underwent metabolism within 3 hours, breaking at the connecting arm, dissociating into the dexamethasone prototype drug and peptide. Therefore, the peptide-drug conjugate designed in the present disclosure can undergo corresponding breaking of the designed connecting arm in vivo, respectively releasing dexamethasone and peptide, exerting their respective therapeutic effects.

In in vitro experiments, the targeting of the conjugate nanomicelle DEX-RGD-TAT-AT7 to integrin protein αᵥβ₃ is preliminarily confirmed at the molecular and cellular levels. In in vitro penetration experiments, the conjugate nanomicelle DEX-RGD-TAT-AT7 can successfully enter cells and has cell barrier penetration activity. DEX-RGD-TAT-AT7 has anti-inflammatory activity and anti-angiogenesis activity.

In in vivo experiments, DEX-RGD-TAT-AT7 exhibits lower hemolytic activity, making it applicable for in vivo experiments. In the mouse ear swelling model. DEX-RGD-TAT-AT7 conjugate micelles inhibits mouse ear swelling, demonstrating in vivo anti-inflammatory effects. In the rat choroidal neovascularization model. DEX-RGD-TAT-AT7 is able to penetrate the ocular barrier to act on the retina and choroid, and has activity in inhibiting choroidal neovascularization, thus playing a certain role in anti-neovascular ocular diseases. The peptide-drug conjugate DEX-RGD-TAT-AT7 designed in the present disclosure can target and penetrate to the fundus with ocular administration, and has dual effects of anti-inflammation and anti-neovascularization, providing a promising treatment strategy for the clinical treatment of neovascular ocular diseases.

The beneficial effects of one or more technical solutions above are as follows:

Compared with the prior art, the present disclosure covalently links the TAT-AT7 peptide, which has penetrating and anti-angiogenic activity, and the RGD peptide, which has targeting integrin activity, with dexamethasone to form a new peptide-drug conjugate. This conjugate continues the anti-inflammatory effect of dexamethasone and has better activity in inhibiting endothelial cell proliferation and inhibiting neovascularization, as well as targeting neovascularization. In addition, the designed connecting arm can break under the action of esterase, ensuring the active effects related to dexamethasone and peptide molecules.

The present disclosure constructs a novel peptide-drug conjugate nanomicelle, which, compared with the prior art, has dual activities of anti-inflammation and anti-angiogenesis, reducing the side effects brought by single-factor treatment of fundus neovascular diseases, and can be used for treating choroidal neovascular ocular diseases through ocular administration, alleviating the burden of poor patient compliance associated with previous vitreous injections. By utilizing the hydrophobicity of dexamethasone and the hydrophilicity of the peptide, spherical nanomicelles are formed in a self-assembled manner, improving the water solubility of dexamethasone. And the novel peptide-drug conjugate nanomicelle can enhance the accumulation and tissue penetration ability of dexamethasone at the site of ocular neovascularization, allowing it to be delivered to the fundus lesions through ocular administration to exert activity. In summary of the above advantages, the conjugate nanomicelles demonstrate strong activity in crossing the ocular barrier and targeting anti-inflammatory and anti-angiogenic effects in in vivo experiments, and can inhibit the progression of neovascular ocular diseases by suppressing the formation of fundus choroidal neovascularization, providing a promising treatment strategy for the clinical treatment of anti-neovascular ocular diseases.

### BRIEF DESCRIPTION OF THE DRAWINGS

The drawings that form part of the description of the present disclosure are used to provide further understanding of the present disclosure. The illustrative embodiments and their descriptions are used to explain the present disclosure and do not constitute improper limitations on the present disclosure.

FIG. 1 is the synthesis route of DEX-SA in Embodiment 1.

FIG. 2 is the synthesis route of DEX-MAL in Embodiment 1.

FIG. 3 is the synthesis route of DEX-RGD, DEX-TAT-AT7, and DEX-RGD-TAT-AT7 in Embodiment 1.

FIG. 4A shows the identification result of DEX-SA in Embodiment 1 by ESI-MS, and FIG. 4B shows the identification result of DEX-SA in Embodiment 1 by ¹H NMR.

FIG. 5A shows the identification result of DEX-MAL in Embodiment 1 by ESI-MS, and FIG. 5B shows the identification result of DEX-MAL in Embodiment 1 by ¹H NMR.

FIG. 6A shows the identification result of DEX-RGD in Embodiment 1 by ESI-Q-TOF, FIG. 6B shows the identification result of DEX-TAT-AT7 in Embodiment 1 by ESI-Q-TOF, and FIG. 6C shows the identification result of DEX-RGD-TAT-AT7 in Embodiment 1 by ESI-Q-TOF.

FIG. 7A shows the critical micelle concentration of DEX-RGD in Embodiment 2, FIG. 7B shows the critical micelle concentration of DEX-TAT-AT7 in Embodiment 2, and FIG. 7C shows the critical micelle concentration of DEX-RGD-TAT-AT7 in Embodiment 2.

FIG. 8A shows the particle size distribution of DEX-RGD in Embodiment 3, FIG. 8B shows the particle size distribution of DEX-TAT-AT7 in Embodiment 3, and FIG. 8C shows the particle size distribution of DEX-RGD-TAT-AT7 in Embodiment 3.

FIG. 9A shows the potential distribution of DEX-RGD in Embodiment 3. FIG. 9B shows the potential distribution of DEX-TAT-AT7 in Embodiment 3, and FIG. 9C shows the potential distribution of DEX-RGD-TAT-AT7 in Embodiment 3.

FIG. 10A is a transmission electron microscope image of DEX-RGD in Embodiment 3, FIG. 10B is a transmission electron microscope image of DEX-TAT-AT7 in Embodiment 3, and FIG. 10C is a transmission electron microscope image of DEX-RGD-TAT-AT7 in Embodiment 3.

FIG. 11A shows, in Embodiment 4, the changes before and after 3 hours of DEX-RGD-TAT-AT7 nanomicelles placed in the aqueous humor detected by HPLC; FIG. 11B shows, at 3 hours of metabolism, the mass spectrometry characterization of the eluent at 7.78 minutes detected by HPLC; FIG. 11C shows, at 3 hours of metabolism, the mass spectrometry characterization of the eluent at 14.9 minutes detected by HPLC; and FIG. 11D shows, at 3 hours of metabolism, the mass spectrometry characterization of the eluent at 16.08 minutes detected by HPLC.

FIG. 12 is the binding response curve of DEX-RGD-TAT-AT7 with integrin αᵥβ₃ in Embodiment 5.

FIG. 13 shows the targeted binding of DEX-RGD-TAT-AT7 to integrin αᵥβ₃ expressed in HUVECs in Embodiment 5.

FIGS. 14A, 14B and 14C show the uptake of DEX-RGD-TAT-AT7 by HCEC cells in Embodiment 6. FIG. 14A is the fluorescence image, FIG. 14B is the cell count curve, and FIG. 14C is the fluorescence intensity histogram, and compared to the blank group, ****P<0.0001; compared to the DEX-RGD group, ^{####}P<0.0001; and compared to the RGD-TAT-AT7 group. ^{&&&&}P<0.0001.

FIGS. 15A, 15B and 15C show the uptake of DEX-RGD-TAT-AT7 by ARPE-19 cells in Embodiment 6. FIG. 15A is the fluorescence image; FIG. 15B is the cell count curve; and FIG. 15C is the fluorescence intensity histogram, and compared to the blank group. ****P<0.0001; compared to the DEX-RGD group, ^{####}P<0.0001; and compared to the RGD-TAT-AT7 group, ^{&&&&}P<0.0001.

FIG. 16 shows the penetration of FITC-DEX-RGD-TAT-AT7 through an in vitro simulated ocular barrier in Embodiment 7.

FIG. 17 shows the effect of DEX-RGD-TAT-AT7 on the proliferation of HUVECs in Embodiment 8 (compared to the DEX group, ***P<0.001, and ****P<0.0001; compared to the DEX-RGD group, ^{####}P<0.0001; compared to the DEX-TAT-AT7 group, ^{&}P<0.05, ^{&&&}P<0.001, and ^{&&&&}P<0.0001, and compared to RGD-TAT-AT7, ^{%%%%}P<0.0001).

FIGS. 18A and 18B show the effect of DEX-RGD-TAT-AT7 on the invasion of HUVECs in Embodiment 8. FIG. 18A is the microscopic image, with a scale bar of 50 μm; and FIG. 18B is the statistical result of cell invasion rate. Compared to the blank group, **P<0.01, and ***P<0.001; compared to the DEX group, ^{##}P<0.01, and ^{####}P<0.0001; compared to the DEX-RGD group, ^{&&&&}P<0.0001; and compared to the DEX-TAT-AT7 group, ^{%%}P<0.01.

FIGS. 19A and 19B show the effect of DEX-RGD-TAT-AT7 on the tube formation of HUVECs in Embodiment 8. FIG. 19A is the microscopic image, with a scale bar of 50 μm; FIG. 19B is the statistic result of total length of tube branches. Compared to the blank group, *P<0.05, **P<0.01, and ***P<0.001; compared to the DEX group, ^{##}P<0.01; compared to the DEX-RGD group, ^{&&}P<0.01; and compared to the DEX-TAT-AT7 group, ^{%}P<0.05.

FIGS. 20A, 20B, 20C, 20D, 20E, and 20F show the release of factors of DEX-RGD-TAT-AT7 that inhibiting inflammatory in Embodiment 9. FIG. 20A is the standard curve of TNF-α, FIG. 20B is the standard curve of IL-6, and FIG. 20C is the standard curve of iNOS. And after 24 hours of incubation with different drugs while stimulating with LPS. FIG. 20D shows the release amounts of TNF-α from BV2, FIG. 20E shows the release amounts of IL-6 from BV2, FIG. 20F shows the release amounts of iNOS from BV2. Compared to the LPS group, ***P<0.001, and ****P<0.0001; compared to the DEX-RGD group, ^{####}P<0.0001; compared to the DEX-TAT-AT7 group, ^{&&}P<0.01, and ^{&&&}P<0.001; and compared to the RGD-TAT-AT7 group, ^{%%%}P<0.001, and ^{%%%%}P<0.0001.

FIGS. 21A and 21B show the release of inhibited NO of DEX-RGD-TAT-AT7 in Embodiment 9. FIG. 21A is the standard curve of NO concentration versus absorbance; FIG. 21B is the NO release amount from BV2 after 24 hours of incubation with different drugs while stimulating with LPS. Compared to the LPS group, ****P<0.0001; compared to the DEX group, ^{##}P<0.01; compared to the DEX-RGD group, ^{&&&&}P<0.0001, compared to the DEX-TAT-AT7 group, ^{%%%}P<0.001; and compared to the RGD-TAT-AT7 group, ^{SSSS}P<0.0001.

FIGS. 22A and 22B show the hemolytic investigation of DEX-RGD-TAT-AT7 in Embodiment 10. In FIG. 22A, from left to right, the drug concentrations are 1000 μg/mL, 500 μg/mL, 250 μg/mL, 100 μg/mL, 50 μg/mL, 25 μg/mL, 10 μg/mL, 5 μg/mL and 2.5 μg/mL, “-” is the negative control group, and “+” is the positive control group. FIG. 22B shows the absorbance of the supernatant is detected by the enzyme marker to count hemolysis rate.

FIG. 23 shows the effect of DEX-RGD-TAT-AT7 on mouse ear swelling in Embodiment 11. Compared to the blank group, ****P<0.0001; and compared to the RGD-TAT-AT7 group, ^{####}P<0.0001 (n=8).

FIG. 24 shows the ocular permeability of DEX-RGD-TAT-AT7 in Embodiment 12.

FIGS. 25A, 25B and 25C show the effect of DEX-RGD-TAT-AT7 on CNV thickness in Embodiment 12 FIG. 25A is the comparison of OCT images of the posterior segment of the normal eye and the posterior segment of the CNV model group when the fundus structure is taken by OCT; FIG. 25B is the OCT images at 7 days and 14 days after different drug eye drop administrations following CNV modeling, and FIG. 25C is the statistical analysis of CNV thickness from OCT. Compared to the Avastin group, *P<0.05, **P<0.01, ***P<0.001, and ns indicates that the difference has no significant meanings (n=3).

FIG. 26 shows the scanning images of RPE-choroidal patches of rats detecting the effect of DEX-RGD-TAT-AT7 on the CNV leakage area in Embodiment 12.

FIG. 27 is the statistical chart of the effect of DEX-RGD-TAT-AT7 on the CNV leakage area detecting the area of neovascularization in Embodiment 12. Compared to the blank group, *P<0.05, and ^{****}P<0.0001, compared to the DEX-RGD group, ^{##}P<0.01, ^{###}P<0.001, and ^{####}P<0.0001; and the Avastin group compared to the ocular administration DEX-RGD-TAT-AT7 group, ns indicates that the difference has no significant meanings (n=3).

### DETAILED DESCRIPTION OF THE EMBODIMENTS

In order to enable those skilled in the art to better understand the technical solutions of the present disclosure, the following will detail the technical solutions of the present disclosure in conjunction with specific embodiments and comparative embodiments

### Embodiment 1: Synthesis and Characterization of the Conjugate DEX-RGD-TAT-AT7

### (1) Synthesis and Characterization of Dexamethasone Succinate (DEX-SA)

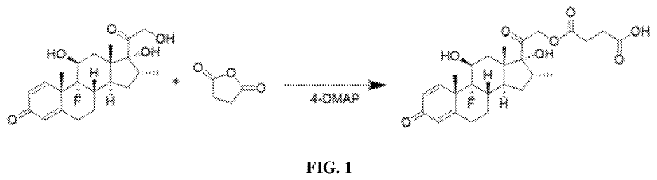

As shown in FIG. 1, 1.5 g of dexamethasone is taken and dissolved in 20 mL of acetone, and stirred until it dissolves. 750 mg of succinic anhydride and 170 mg of DMAP (4-dimethylaminopyridine) are then taken and dissolved in the reaction system, and stirred at room temperature for 4 hours. The reaction is monitored using thin-layer chromatography (TLC), with a developing agent ratio of dichloromethane:methanol=9:1. After monitoring that the reaction is nearly complete, a rotary evaporator is used to rotate and dry the reaction solution, and a yellowish oily viscous substance can be seen adhering to the bottom of the bottle.

Anhydrous ethanol is added dropwise to the product obtained after rotating and drying, shaking while adding until the oily viscous substance re-dissolves, then distilled water at 60° C. is added dropwise until sediment reoccurs and suspends in the solution. At this time, the ratio of distilled water to anhydrous ethanol is approximately 29:71. The solution is heated with a spherical condenser to reflux until the precipitate is completely dissolved, and the solution is immediately transferred to 4° C., and stood for 24 hours.

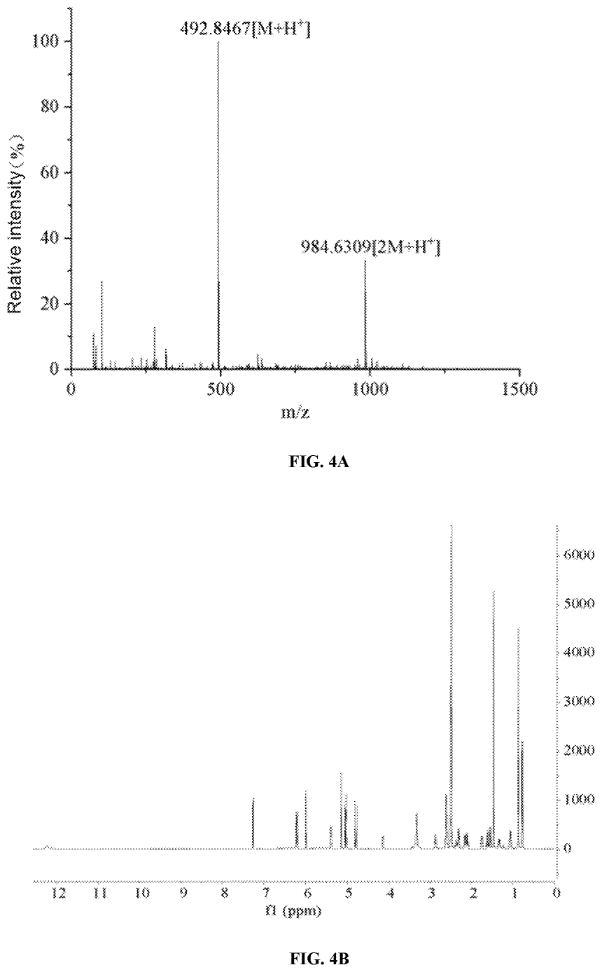

The solution is removed, and a white precipitate can be seen. A suction filtration device is used to filter out the liquid, the precipitate is obtained, which is washed 3 times with 3 mL of 25% ethanol, and dried overnight in a 50° C. oven. The resulting white solid powder is the product of this step. The product is weighted to calculate the yield, high-performance liquid chromatography (HPLC) is used to detect its purity, and ¹H-NMR and ESI-MS are used to calculate its molecular mass and confirm its structure, the results are shown in FIGS. 4A and 4B.

### (2) Synthesis and Characterization of Dexamethasone-Maleimide Conjugate (DEX-MAL)

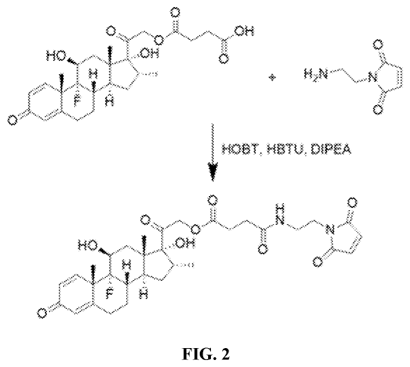

As shown in FIG. 2, 250 mg of the previous product DEX-SA, 290 mg of 2-(7-nitrobenzotriazole)-N,N,N′,N′-tetramethylurea hexafluorophosphate (HATU), and 70 mg of 1-hydroxybenzotriazole (HOBT) are taken and dissolved in 10 mL of dichloromethane, then 450 μL of N,N-diisopropylethylamine (DIPEA) is added to the reaction system, stirring at room temperature for 1 hour to dissolve. Then 125 mg of N-(2-aminoethyl) maleimide trifluoroacetate (MAL) is added to the reaction system and continuously stirred at room temperature for 12 hours. TLC monitors that the reaction is nearly complete, then a rotary evaporator is used to rotate and dry the reaction solution.

The rotated and dried reactant is re-dissolved in 3 mL of dichloromethane, the product is purified using a silica gel chromatographic column, with the eluent being methanol:dichloromethane=1:25. The product is collected and freeze-dried, and the dexamethasone-maleimide conjugate DEX-MAL can be obtained and weighed to calculate the yield. Purity of the dexamethasone-maleimide conjugate DEX-MAL is detected using HPLC, and ¹H-NMR and ESI-MS are used to calculate its molecular mass and confirm its structure, results are shown in FIGS. 4A and 4B.

### (3) Synthesis and Characterization of Peptide-Dexamethasone Conjugate

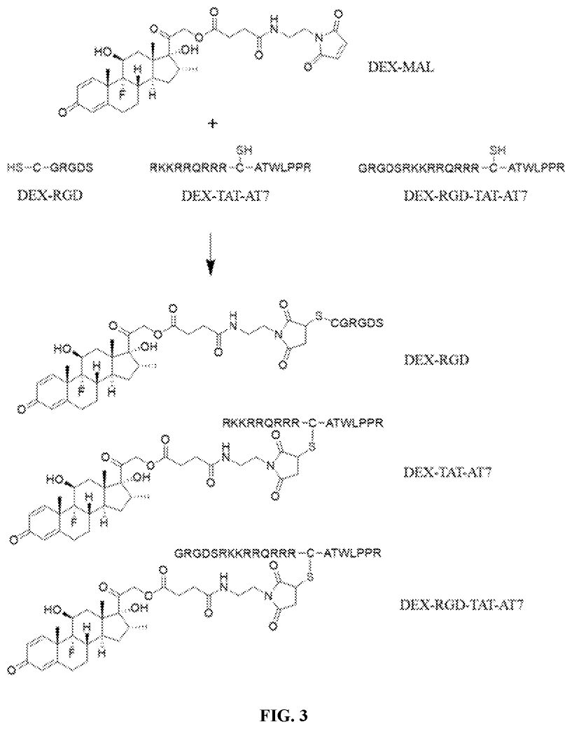

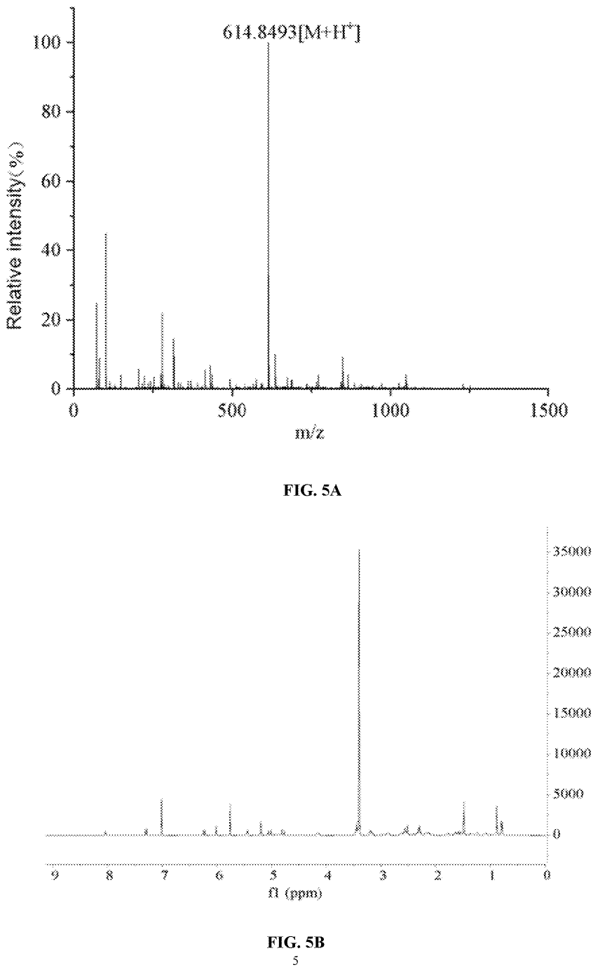

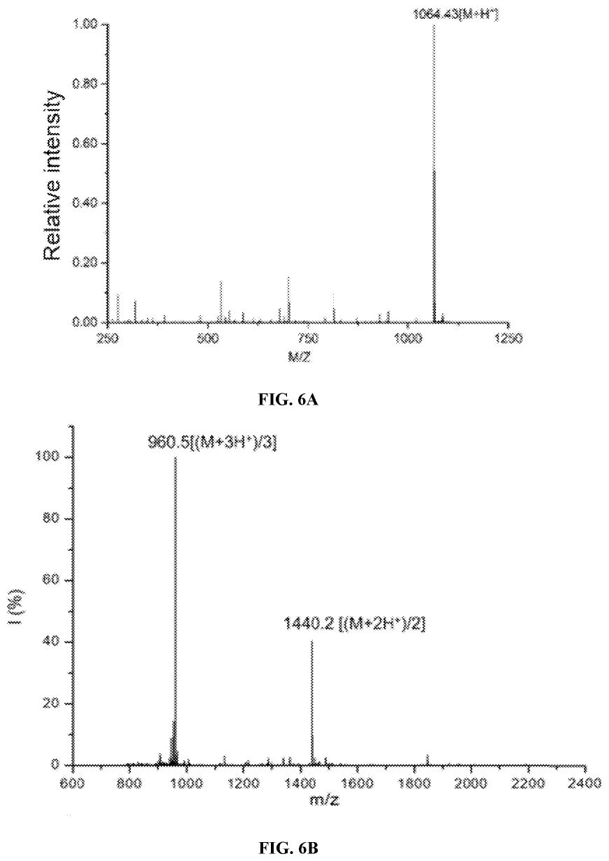

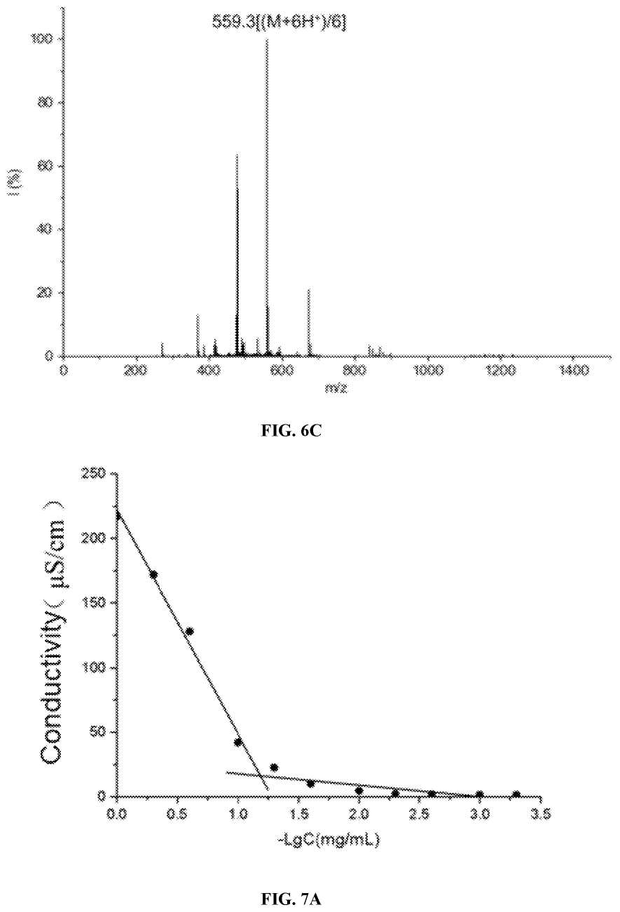

DEX-RGD: 20 mg of the dexamethasone-maleimide conjugate (DEX-MAL) is taken and dissolved in 2 mL of acetonitrile, and 25 mg of the peptide CGRGDS (as shown in SEQ ID NO. 1, where C is a thiolated cysteine) is taken and dissolved in 2 mL of PBS. The two solutions are mixed and stirred at room temperature for 2 hours, as shown in FIG. 3. Analytical HPLC is used to monitor the reaction, with mobile phase A being deionized water+0.1% TFA, and mobile phase B being acetonitrile+0.1% TFA. The elution gradient is: 0 minute, 90% A, 10% B; 10 minutes, 60% A, 40% B; 20 minutes, 40% A, 60% B; and 25 minutes, 20% A, 80% B. The ultraviolet detection wavelength is 220 nm, the injection volume is 50 pt, and the flow rate is 1 mL/min. It is confirmed that the raw material peak has essentially disappeared, and that the area of the new product peak accounts for over 90% area of the total peak, indicating that the reaction is essentially complete. The reaction solution is purified using preparative HPLC, with mobile phase A being deionized water+0.1% TFA, and mobile phase B being acetonitrile+0.1% TFA. The elution gradient is: 0 minute, 90% A, 10% B; 30 minutes, 60% A, 40% B; 60 minutes, 40% A, 60% B; and 80 minutes, 20% A, 80% B. The ultraviolet detection dual wavelengths are 220 nm and 240 nm, and the flow rate is 6 mLImin. ESI-Q-TOF mass spectrometry analysis is performed on the collected main peak eluent from the preparative HPLC analysis to obtain the molecular mass of the eluent, with results shown in FIG. 6A.

DEX-TAT-AT7: 12 mg of dexamethasone-maleimide conjugate (DEX-MAL) is taken and dissolved in 1 mL of acetonitrile, and 40 mg of the peptide TAT-AT7 (RKKRRQRRRCATWLPPR, as shown in SEQ ID NO. 5, where C is a thiolated cysteine) is taken and dissolved in 2 mL of PBS. The two solutions are mixed and stirred at room temperature for 2 hours, as shown in FIG. 3. The reaction monitoring analysis and product purification preparation method of DEX-TAT-AT7 are the same as that of DEX-RGD. The obtained solution is freeze-dried and weighed to calculate the yield. ESI-Q-TOF is used to determine its molecular mass, with results shown in FIG. 6B.

DEX-RGD-TAT-AT7: 12 mg of dexamethasone-maleimide conjugate (DEX-MAL) is taken and dissolved in 1 mL of acetonitrile, and 50 mg of the peptide RGD-TAT-AT7 (GRGDSRKKRRQRRRCATVLPPR, as shown in SEQ ID NO. 4, where C is a thiolated cysteine) is taken and dissolved in 2 mL of PBS. The two solutions are mixed and stirred at room temperature for 2 hours, as shown in FIG. 3 The reaction monitoring analysis and product purification preparation method of DEX-RGD-TAT-AT7 are the same as that of DEX-RGD. The obtained solution is freeze-dried and weighed to calculate the yield. ESI-Q-TOF is used to determine its molecular mass, with results shown in FIG. 6C.

### Embodiment 2: Determination of Critical Micelle Concentration of Peptide-Dexamethasone Conjugate

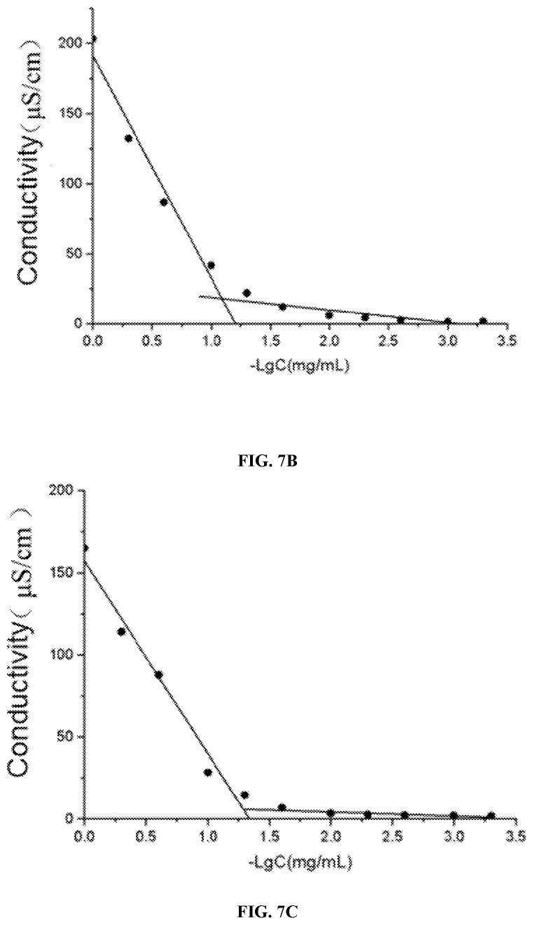

The conductivity method is used to determine the critical micelle concentration of the prepared nanomicelles. Three types of conjugated nanomicelles are prepared into nanomicelle solutions with concentration gradients of 1 mg/mL, 0.5 mg/mL, 0.25 mg/mL, 0.1 mg/mL, 0.05 mg/mL, 0.025 mg/mL, 0.01 mg/mL, 0.005 mg/mL, 0.0025 mg/mL, 0.001 mg/mL, and 0.0005 mg/mL, respectively, with 20 mL of each concentration. The conductivity meter probe is inserted into the solutions to record the conductivity values when the reading stabilizes. The measured conductivity inflection point is the critical micelle concentration, with results shown in FIGS. 7A, 7B and 7C. The critical micelle concentrations of nanomicelles DEX-RGD. DEX-TAT-AT7, and DEX-RGD-TAT-AT7 are 0.0494 mg/mL, 0.0808 mg/mL, and 0.0650 mg/mL, respectively. This indicates that all three conjugates can self-assemble into nanomicelles in aqueous solution at relatively low concentrations.

### Embodiment 3: Preparation and Physical-Chemical Property Characterization of Peptide-Dexamethasone Conjugate Nanomicelles

The freeze-dried powders of DEX-RGD, DEX-TAT-AT7, and DEX-RGD-TAT-AT7 are taken and dissolved in deionized water to prepare aqueous solutions with concentration gradients of 4 mg/mL, 2 mg/mL, 1 mg/mL, 0.5 mg/mL, 0.25 mg/mL, 0.125 mg/mL, 0.0625 mg/mL, 0.03125 mg/mL, 0.016 mg/mL, and 0.008 mg/mL, respectively. Nanomicelles are prepared using a vortex ultrasound method. First, the conjugate is vortexed at low speed for 10~30 seconds and then sonicated for 10 minutes. The prepared solutions are filtered using a 0.22 μm microporous filter membrane.

The morphology of the prepared nanomicelles is observed using transmission electron microscopy. Nanomicelle solutions of DEX-RGD. DEX-TAT-AT7, and DEX-RGD-TAT-AT7 at a concentration of 4 mg/mL are prepared. Tweezers are used to clamp copper grids and carefully immerse them into the nanomicelle solutions, ensuring that both sides of the copper grid membrane are loaded with the sample. After the solution on the copper grid membrane has dried sufficiently, the copper grids with the sample are placed in front of the sample rod and their morphologies are observed wider a transmission electron microscope to assess whether the nanomicelles have been successfully prepared.

The nanomicelle solutions of DEX-RGD, DEX-TAT-AT7, and DEX-RGD-TAT-AT7 with a concentration of 4 mg/mL are filtered through a 0.22 μm microporous filter membrane, and then added into a ZETA potential sample cell, and the particle size and potential of the nanomicelle solutions are measured using a Malvern particle size analyzer.

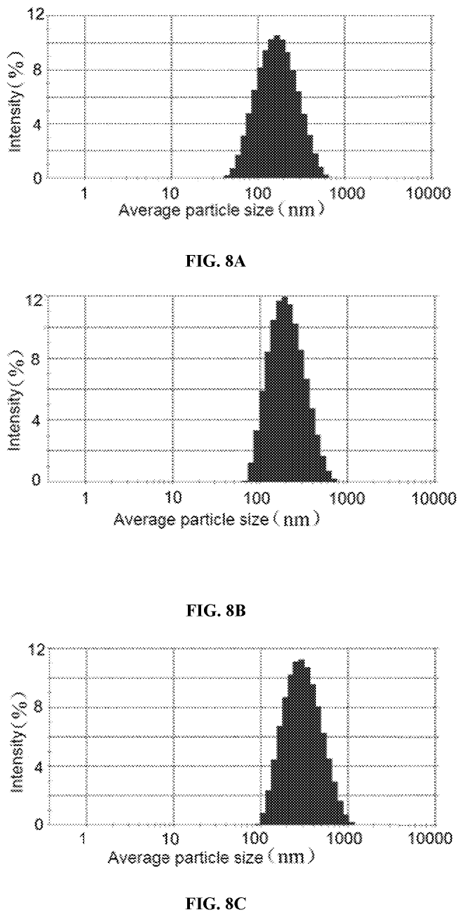

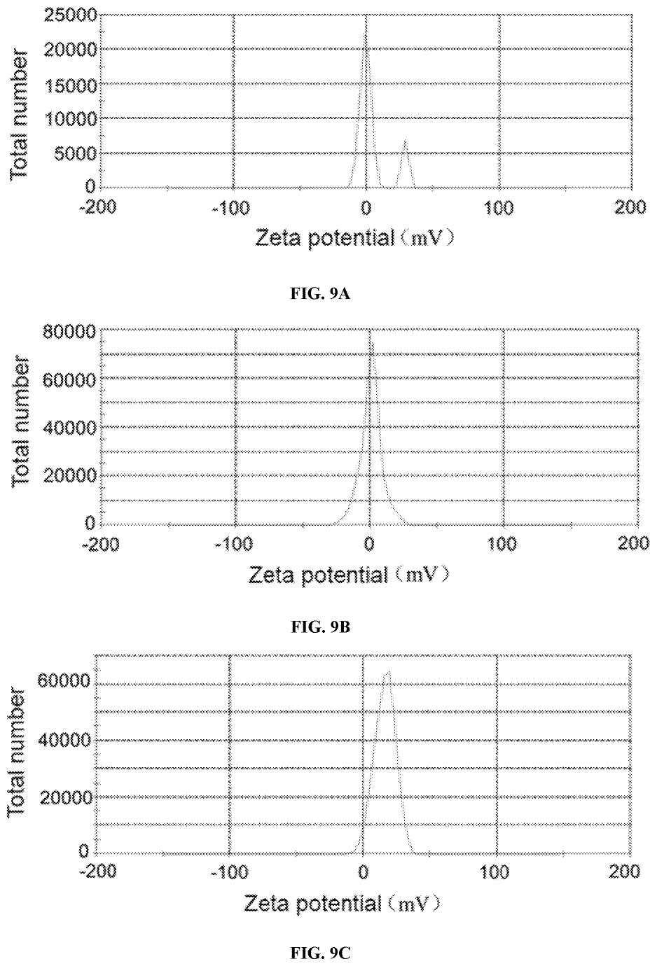

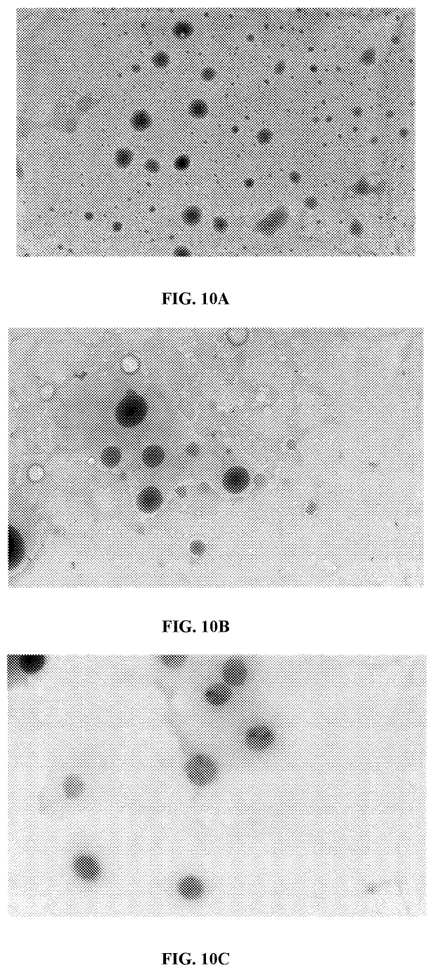

The results of particle size analysis and Zeta potential measurement are shown in FIGS. 8A, 8B, 8C, 9A, 9B, 9C, 10A, 10B and 10C, indicating that the particle sizes of the three types of nanomicelles are all around 100-200 nm and exhibit positive charge

### Embodiment 4: Dissociation of Nanomicelles in In Vitro Aqueous Humor

### (1) Establishment of Standard Curve

DEX-RGD-TAT-AT7 is prepared into a concentrated solution of 50 mmol/L, then the concentrated solution is diluted into solutions with concentration gradients of 50 mmol/L, 40 mmol/L, 30 mmol/L, 20 mmol/L, 10 mmol/L, 8 mmol/L, 6 mmol/L, 4 mmoL/L, 2 mmol/L, and 1 mmol/L, respectively, 10 μL of each concentration of DEX-RGD-TAT-AT7 is taken and placed in 1.5 mL EP tubes, 90 μL of rabbit aqueous humor is added into the EP tubes, and then 300 μL of protein precipitant (acetonitrile:methanol=9:1) is immediately added. The EP tubes are transferred to a −80° C. freezer and frozen for 30 minutes to precipitate the proteins in the aqueous humor. Then they are centrifuged at 12000 r/min for 30 minutes, and the supernatants are collected, 800 μL of dichloromethane is added for extraction, the upper layer of liquid is taken, and filtered through a 0.22 μm filter membrane, and detected using analytical HPLC. The drug content is quantified based on the peak area of different concentration solutions in HPLC.

### (2) Determination of Dissociation of DEX-RGD-TAT-AT7 Nanomicelles in In Vitro Aqueous Humor Using HPLC

DEX-RGD-TAT-AT7 is prepared into a 2 mmol/L aqueous solution, 100 μL of DEX-RGD-TAT-AT7 is taken and placed in a 1.5 mL EP tube, and 900 μL of aqueous humor is added 100 μL of the mixture is sampled at moments: 0 minute, 2 minutes, 5 minutes, 10 minutes, 20 minutes, 30 minutes, 45 minutes, 60 minutes, 90 minutes, and 120 minutes, respectively, and 300 μL of protein precipitant is immediately added after sampling. The subsequent detection method is the same as that in (1). The main peak elution liquid of DEX-RGD-TAT-AT7 samples taken at different moments is characterized using ESI-MS, and the peak time and peak area are analyzed.

The results are shown in FIGS. 11A, 11B, 11C and 11D. Through HPLC monitoring, most of the drug underwent metabolism within 3 hours, with DEX-RGD-TAT-AT7 breaking at the connecting arm, dissociating into dexamethasone prototype drug and peptides. This indicates that DEX-RGD-TAT-AT7 prepared in Embodiment 1 can undergo corresponding breaking of the designed connecting arm in in vivo, releasing dexamethasone and peptides, exerting their respective therapeutic effects.

### Embodiment 5: Targeting of DEX-RGD-TAT-AT7 to Integrin αᵥβ₃

(1) Affinity of DEX-RGD-TAT-AT7 to Integrin αᵥβ₃

A micro-scale thermophoresis instrument (MST) is used to detect the interaction between DEX-RGD-TAT-AT7 and integrin protein. First, the protein sample is prepared. The 10×HBS-EP buffer concentrate is diluted 10 times with double-distilled water to 1×HBS-EP buffer, then an appropriate amount of integrin αᵥβ₃ protein solution is added into the 1-HBS-EP buffer to dilute the protein concentration to 100 nmol/L. 2 μL of the dye His-Tag Labeling Kit RED-tris-NTA is taken and added into 98 μL of HBS-EP and mixed well to obtain a final volume of 100 μL of His-Tag Labeling Kit RED-tris-NTA. 90 μL of the integrin αᵥβ₃ solution and 90 μL of the dye solution are taken and mixed well and stood at room temperature for 30 minutes to allow the protein to fully bind with the dye. Then the mixture is centrifuged at 15000-g for 10 minutes, the supernatant is taken and transferred to a new EP tube, and stored at −20° C. for later use.

DEX-RGD-TAT-AT7 is prepared into a concentrated solution of 5 mmol/L using HBS-EP, and gradiently diluted to 2.5 mmol/L, 1.25 mmol/L, 6.25×10⁻¹ mmol/L, 3.125×10⁻¹ mmol/L, 1.563×10⁻¹ mmol/L, 7.813×10⁻² mmol/L, 3.906×10⁻² mmol/L, 1.953×10⁻² mmol/L, 9.766×10⁻³ mmol/L, 4.883×10⁻³ mmol/L, 2.441×10⁻³ mmol/L, 1.221×10⁻³ mmol/L, 6.104×10⁻⁴ mmol/L, 3.052×10⁻⁴ mmol/L, and 1.526×10⁻⁴ mmol/L, respectively, 10 μL of each concentration of DEX-RGD-TAT-AT7 is taken and placed in 0.1 mL EP tubes, then 10 μL of the labeled RED-tris-NTA integrin αᵥβ₃ solution is added into each tube, then they are gently blown by pipetting gun for 10 times to mix well, and incubated at room temperature for 20 minutes. The capillary is inserted into the solution of EP tubes, and the capillary is gently tilted to take samples, avoiding the generation of bubbles in the tubes, and the capillary is placed on the Monolith instrument for detection.

(2) Binding of DEX-RGD-TAT-AT7 to Integrin αᵥβ₃ Expressed by HUVECs

Logarithmically growing HUVEC cells are taken, digested and centrifuged, resuspended and counted in complete culture medium, the cell density is adjusted to 2×10⁵ cells/mL, and the cells are plated in a confocal dish and incubated at 37° C. in dark overnight. After the cells adhere, the original culture medium is removed, medium containing FITC-labeled DEX-RGD-TAT-AT7 conjugated nanomicelles is added to achieve a drug concentration of 20 μmol/L. And a control group is set up by replacing the original culture medium with drug-free medium and incubated at 37° C. for 30 min. The dish is removed and operated under room temperature and light protection. The culture medium is discarded, 1 mL of PBS is added to gently wash 3 times, then 800 μL of 4% paraformaldehyde fixative is added, and the cells are fixed at room temperature for 30 min, and then PBS is used to gently wash 3 times. A PBS solution containing 1% BSA and 0.5% Triton X-100 is prepared to block nonspecific binding. 1 ml of the PBS solution is added to a dish and blocked for 30 minutes at room temperature. The APC conjugated antibody is diluted 25 times with blocking solution, 250 μL of diluted antibody is added to each dish, and incubated at 4° C. in dark overnight. The next day, the liquid within the dish is discarded, PBS is used to carefully wash three times, 400 μL DAPI staining solution is added into each dish to stain in dark at room temperature for 10 minutes, and then PBS is used to carefully wash three times, and finally 100 μL PBS is added into the dish to infiltrate cells, and one drop of anti-fluorescence quencher is dropped into the dish. The binding sites of fluorescent drugs on cells are observed and photographed under a laser confocal microscope.

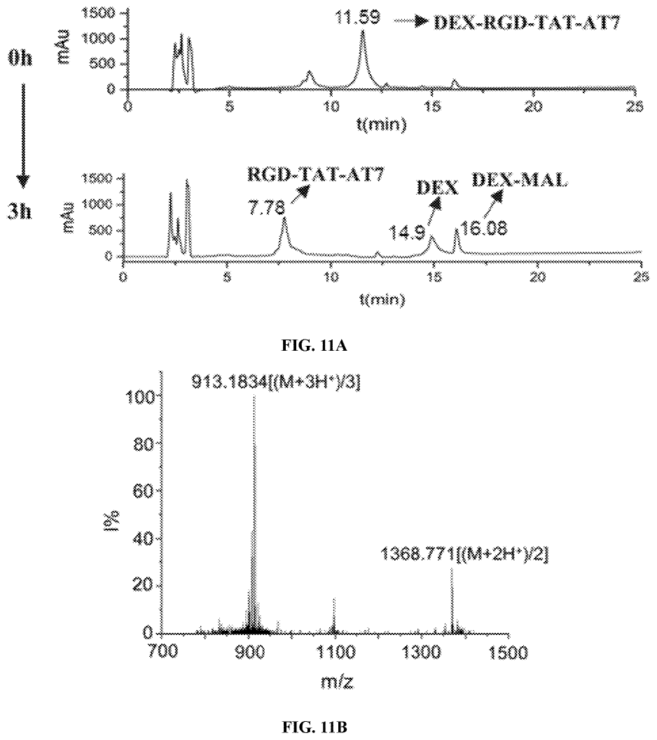

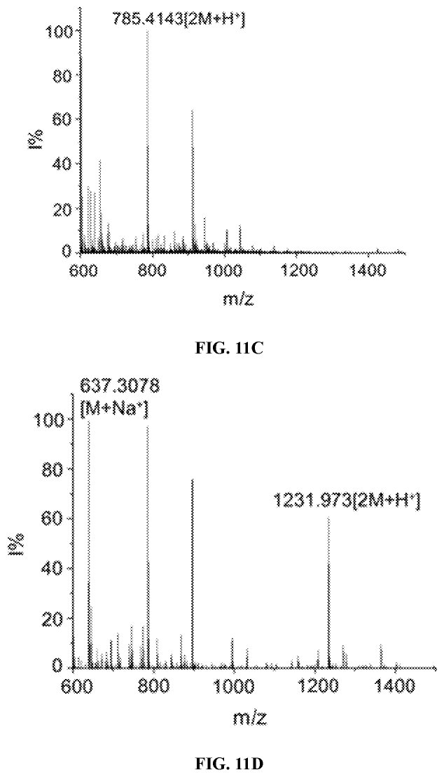

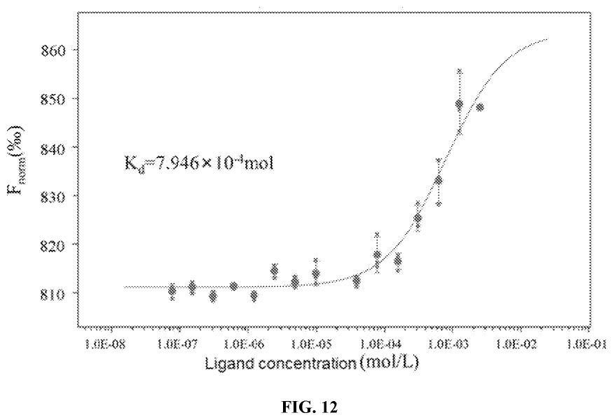

The binding response curve of DEX-RGD-TAT-AT7 to integrin αᵥβ₃ is shown in FIG. 12, indicating that the conjugate nanomicelle DEX-RGD-TAT-AT7 has targeting ability towards integrin αᵥβ₃, with an affinity (Kd) of 7.946×10⁻⁴ mol.

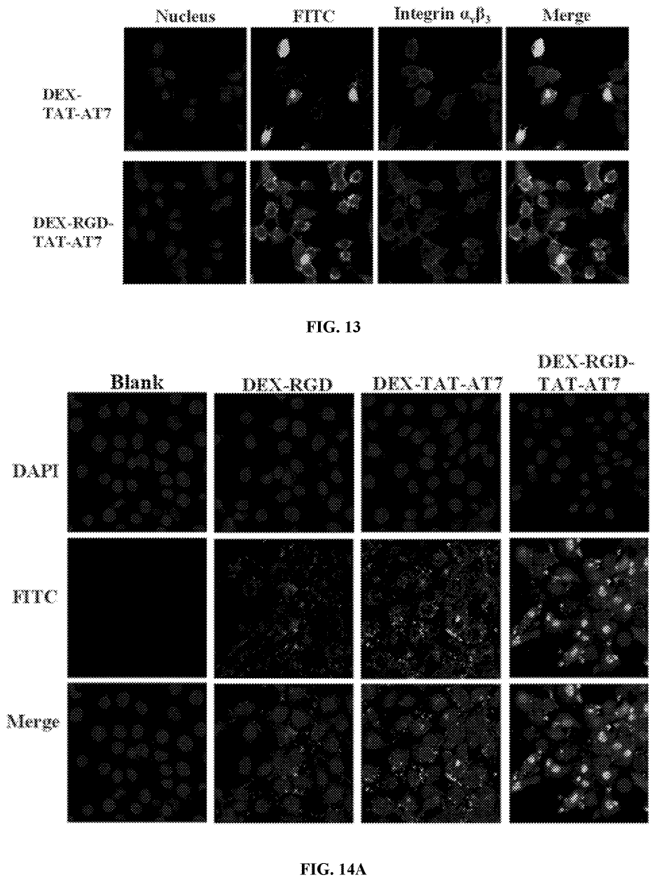

The binding results of DEX-RGD-TAT-AT7 to integrin αᵥβ₃ expressed by HUVECs are shown in FIG. 13. FITC-DEX-RGD-TAT-AT7 has a certain degree of co localization with integrin αᵥβ₃ expressed on HUVECs, while FITC-DEX-TAT-AT7 without RGD coupling partially enters cells and does not bind to the surface of cell membrane. The above results indicate that DEX-RGD-TAT-AT7 can bind to the receptor integrin αᵥβ₃ on the surface of HUVECs membrane.

### Embodiment 6: The Cell Uptake Ability of DEX-RGD-TAT-AT7

### (1) Observation of Cell Uptake by Laser Confocal Microscopy

Logarithmic growth phase HCEC cells and ARPE-19 cells are taken, digested and centrifuged, and resuspended in complete culture medium. The cell density is adjusted to 3×10⁵ cells/mL, 1 mL of cell suspension is added to the confocal dish, and incubated overnight at 37° C. After the cells adhere, the culture medium containing the conjugated nanomicelles is prepared, grouping as FITC-DEX-RGD, FITC-DEX-TAT-AT7, and FITC-DEX-RGD-TAT-AT7. The original culture medium is removed from the dish and replaced with the drug-containing medium. A control group with basic culture medium is set up. Then they are incubated at 37° C. for 4 hours, and then the dish is removed and operated under room temperature and light protection. The culture medium is discarded, and 1 mL of PBS is added into the dish to gently wash 3 times Then 800 μL of 4% paraformaldehyde fixative is added, and cells are fixed at room temperature for half an hour. After fixation, gently wash with PBS is performed for 3 times again. 400 μL of DAPI staining solution is added to each dish to stain for 10 minutes. After staining, gently wash with PBS is performed for 3 times and about 100 μL of PBS is added to saturate the bottom of the dish. Finally, photos are taken under a laser confocal microscope and the fluorescence intensity is analyzed using Image J software.

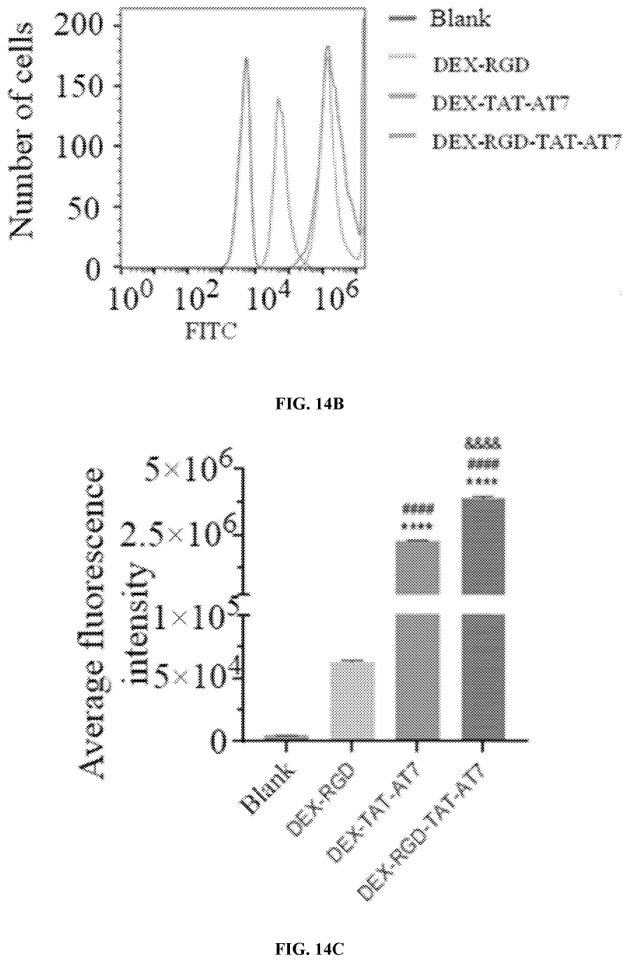

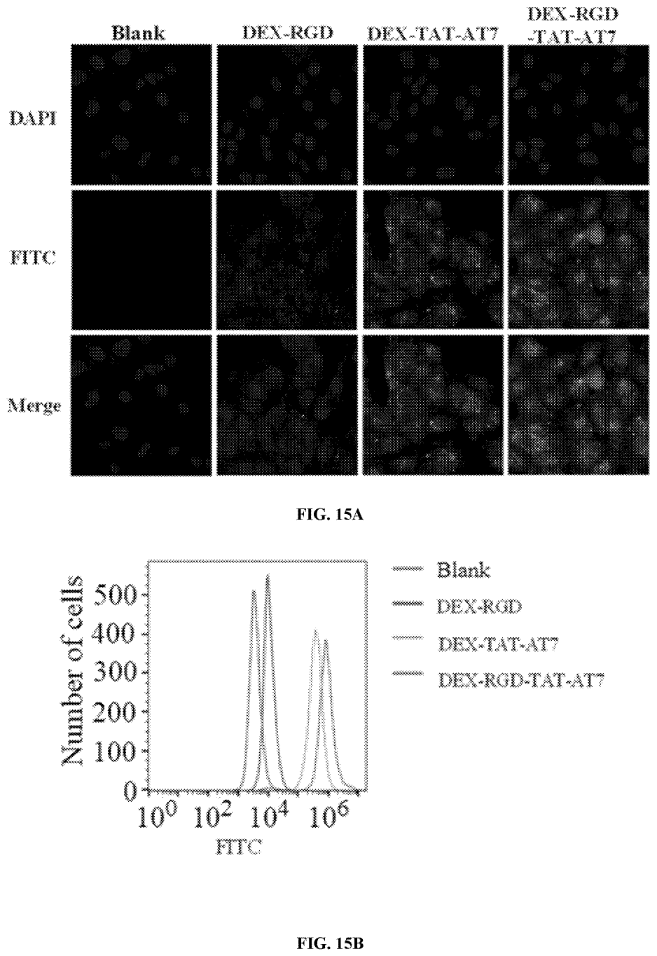

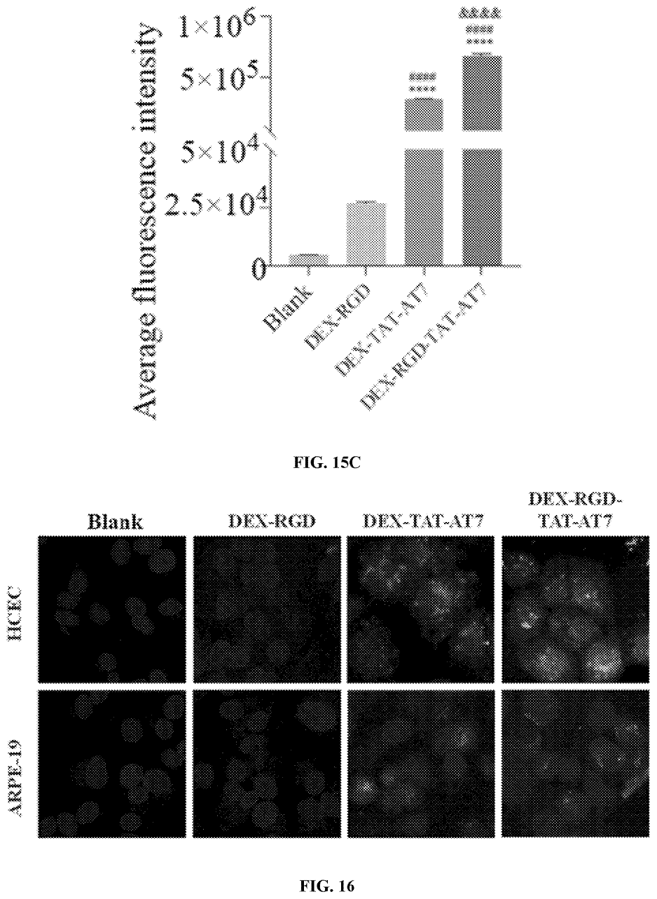

The qualitative observation results of the cell uptake ability of DEX-RGD-TAT-AT7 are shown in FIG. 14A and FIG. 15A. After co-incubating the FITC-conjugated drug with the cells for 1 hour, both HCEC cells and ARPE-19 cells showed significant uptake of DEX-TAT-AT7 and DEX-RGD-TAT-AT7, with the DEX-RGD-TAT-AT7 group showing the highest uptake, while the DEX-RGD group had very weak fluorescence intensity.

### (2) Detection of Cell Uptake by Flow Cytometry

Logarithmic growth phase HCEC cells and ARPE-19 cells are taken, digested and centrifuged, and resuspended by complete culture medium to adjust the cell density to 5×10⁵ cells/mL, and the cells are added into a six-hole plate, and incubated overnight at 37° C. The drug administration part is the same as that of the quantitative uptake experiment. After drug administration, the cells are incubated for 4 hours. Then the six-hole plate is removed, 400 μL of trypsin is added into each hole for digesting for 2~3 minutes. The digested cells are transferred to a 15 mL centrifuge tube and centrifuged at 2000 r/min for 3 minutes. After centrifugation, 2 mL of PBS is added into the cell precipitate and gently vortexed to mix, then they are continuously centrifuged at 2000 r/min for 3 minutes. The above PBS washing steps are repeated twice, and 400 μL of PBS is finally added to resuspend the cell precipitate. The cells are filtered through a 70 μm cell strainer and transferred to a flow cytometry tube. The differences in fluorescence intensity among the groups are detected using a flow cytometer, with a cell count event of 10.000 and a flow rate of 10 μL/s. The results are finally analyzed using flow jo software.

The quantitative results of the cell uptake ability of DEX-RGD-TAT-AT7 are shown in FIG. 14B and FIG. 15B The fluorescence intensity of the FITC-DEX-RGD-TAT-AT7 group in HCEC cells and ARPE-19 cells reached 60.64 times and 24.95 times that of the FITC-DEX-RGD group, respectively, with differences having significant meanings compared to the DEX-TAT-AT7 group and DEX-RGD group. The above results indicate that DEX-RGD-TAT-AT7 has a stronger cell uptake ability, and DEX-RGD-TAT-AT7 may have the best permeability among the three types of conjugated nanomicelles.

### Embodiment 7: DEX-RGD-TAT-AT7 Penetrates In Vitro Cell Barriers

Logarithmic growth phase HCEC cells and ARPE-19 cells are taken, digested and centrifuged, and resuspended by MEM complete culture medium and DMEM/F12K complete culture medium to adjust a cell density to be 2×10⁵ cells/mL, respectively. Two 24-hole plates are prepared, the cell culture inserts are placed in the holes with tweezers, the two types of cells at a volume of 400 μL per hole are inoculated into the 24-hole plates, and incubated at 37° C. for 24 hours Then the 24-hole plates coated with HCEC cells are removed, the original culture medium is aspirated, and 400 μL of FITC-labeled conjugate nanomicelles is added and grouped as FITC-DEX-RGD, FITC-DEX-TAT-AT7, and FITC-DEX-RGD-TAT-AT7, to achieve a final drug concentration of 20 μmol/L. After incubating at 37° C. for 4 hours, the inserts with incubated FITC conjugates HCEC cells and ARPE-19 cells are taken out and placed in a new 6-hole plate, and DMEM/F12K culture medium is added for mixed culture, ensuring that each hole contains one administered HCEC cell insert and one unadministered ARPE-19 cell insert. Then the new 6-hole plate is continued to incubate at 37° C. for 4 hours. After incubating, the original culture medium is aspirated from the 6-hole plate, and PBS is used to gently wash the 6-hole plate for 3 times, then 1 mL of 4% paraformaldehyde fixative is added to fix the cells at room temperature for 30 minutes. Then PBS is used again to gently wash for 3 times, and 500 μL of DAPI staining solution is added to each hole to stain at room temperature in the dark for 10 minutes. Then PBS is used to gently wash for 3 times, the cell inserts are carefully removed with tweezers. The side of the cell insert with cells slowly covers on microscope slide, which is dropped with anti-fluorescence quencher, avoiding bubble formation. Finally, the drug fluorescence intensity on the two types of cell inserts is observed under a laser confocal microscope.

The results of DEX-RGD-TAT-AT7 penetrating the in vitro cell barrier are shown in FIG. 16. Through the laser confocal microscopy, it can be seen that after co-incubating the FITC conjugated drug with the HCEC cell-coated cell inserts for 4 hours, both the DEX-TAT-AT7 group and the DEX-RGD-TAT-AT7 group exhibited good cellular uptake capability. After continuing to co-incubate with ARPE-19 cells for 4 hours, the uptake in the DEX-RGD-TAT-AT7 group is higher compared to the DEX-TAT-AT7 group. The above results indicate that DEX-RGD-TAT-AT7 can successfully penetrate cells through endocytosis and, after exocytosis, penetrate into other cells to achieve cell barrier penetration, and it has better permeability compared to DEX-TAT-AT7, suggesting that DEX-RGD-TAT-AT7 may have a certain ability to penetrate the ocular barrier.

### Embodiment 8: Evaluation of In Vitro Anti-Angiogenic Activity of DEX-RGD-TAT-AT7

### (1) the Effect of DEX-RGD-TAT-AT7 on Endothelial Cell Proliferation

The CCK-8 method is used to determine the cell viability after drug administration. After digesting the cells, the cells are resuspended by complete culture medium. 10 μL of the cell suspension is taken onto a cell counting plate and the cells are counted by a microscope, following the rule of “count upper and left, and not count lower and right” (i.e., when the cells are observed by the microscope and when cells are located at upper boundary, lower boundary, left boundary, and right boundary, the cells located at the upper boundary and the left boundary are counted, and the cells located at the lower boundary and the right boundary are not counted). The total cell number in four large squares of the cell counting plate is divided by four to obtain the cell concentration (10⁴ cells/mL). The cell concentration is then diluted to 5×10⁴ cells/mL and plated in a 96-hole plate. A blank group without cells is set up, a basic culture medium without cells is only added. After 24 hours, a culture medium containing conjugated nanomicelles is prepared using F12K basic culture medium and grouped as DEX. DEX-RGD, DEX-TAT-AT7, RGD-TAT-AT7, and DEX-RGD-TAT-AT7, the concentration gradient of each group of conjugated nanomicelles is set as 320 μmol/L, 160 μmol/L, 80 μmol/L, 40 μmol/L, 20 μmol/L, and 10 μmol/L, respectively, and they are filtered through a 0.22 μm microporous filter. A control group is also set up, and basic culture medium without drugs is added into holes of the control group. The culture medium is aspirated from the 96-hole plate and replaced the culture medium with 100 μL of drug-containing culture medium, then the 96-hole plate is incubated in 37° C. incubator for 24 hours. The original culture medium is aspirated, a culture medium containing 10% CCK-8 is prepared with F12K basic culture medium, with 100 μL per hole, and the culture medium containing 10% CCK-8 is incubated at 37° C. for 1-4 hours. Finally, the 96-hole plate is taken out and the optical density (OD value) is measured and read at 450 nm of a microplate reader. The following formula is used to calculate cell viability:

$$Cell inhibition rate ( \% ) = 1 - ( OD of drug - adminstration group - OD of blank group ) / ( OD of control group - OD of blank group ) \times 100 \%$$

The results of the effect of DEX-RGD-TAT-AT7 on endothelial cell proliferation are shown in FIG. 17. The DEX group and DEX-RGD group have no significant effect on HUVECs cell proliferation, with cell growth inhibition rates around 10%. However, the RGD-TAT-AT7 group. DEX-TAT-AT7 group, and DEX-RGD-TAT-AT7 group all exhibit a certain concentration-dependent anti-proliferative activity on HUVECs, with the cell proliferation inhibition rate increasing with the increase in drug concentration, and the DEX-RGD-TAT-AT7 group shows a higher cell proliferation inhibition rate. The above results indicate that compared to DEX-RGD. DEX-TAT-AT7, and RGD-TAT-AT7. DEX-RGD-TAT-AT7 has better activity in inhibiting endothelial cell proliferation.

### (2) The Effect of DEX-RGD-TAT-AT7 on Cell Invasion

One day in advance, the matrix gel is placed at 4° C. to melt, and before the experiment, the matrix gel is placed on the ice bucket. The matrix gel is prepared in the ultra-clean bench, and the matrix gel is diluted with F12K basic culture medium at a ratio of F12K:matrix gel=5:1, and mixed them well. A 24-hole plate is taken, a tweezer is used to place the small chambers into the 24-hole plate to aspirate the diluted matrix gel. A small chamber is added to 50 μL of each hole while shaking to evenly cover the bottom of the small chambers with the matrix gel. The 24-hole plate coated with matrix gel is transferred to a 37° C. incubator and incubated for 1-2 hours until the matrix gel solidifies. 20% serum is added to the basic culture medium to obtain a 20% complete culture medium, the logarithmic phase HUVECs are digested and resuspended, and the cells are counted and the cell density is adjusted to 3×10⁵ cells/mL. The 24-hole plate is removed, a 10 μL pipette gun is used to aspirate the unsolidified matrix gel, 600 μL of 20% complete culture medium is added to the Transwell lower chamber, then 100 μL of the cell suspension is added in the small chamber, and then the 24-hole plate is transferred to a 37° C. incubator to continue culturing for 24 hours. 100 μL of the culture medium containing the conjugate nanomicelles is added to each hole in the small chamber, grouping as DEX group, DEX-RGD group, DEX-TAT-AT7 group, RGD-TAT-AT7 group, and DEX-RGD-TAT-AT7 group, making the final concentration of the nanomicelles 40 μmol/L. Then a control group is set up by adding 100 μL of basic culture medium to the small chamber and continuing to culture for 24 hours. The fixative fluid is prepared at a ratio of methanol:ice acetic acid=3:1. Then the small chambers are removed, and washed with pre-cooled PBS for three times. 600 μL of fixative fluid is added to a clean 24-hole plate, and the small chambers are transferred to the holes containing the fixative fluid, and fixed at room temperature for 30 minutes. Then the small chambers are washed with PBS for three times, and 600 μL of 0.1% crystal violet is added to the lower chamber to stain at room temperature for 30 minutes. Then PBS is used to wash again for three times, the cells and matrix gel are gently wiped from the inner side of the small chambers with a cotton swab, and photos under a bright field are taken using an inverted fluorescence microscope to analyze the number of cells that penetrate through the small chambers. The following formula is used to calculate the cell migration rate:

$$Cell migration rate ( \% ) = Number of cells in the drug - administration group / Number of cells in the control group \times 100 \%$$

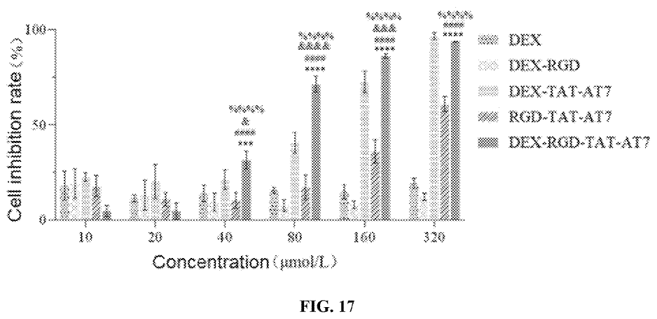

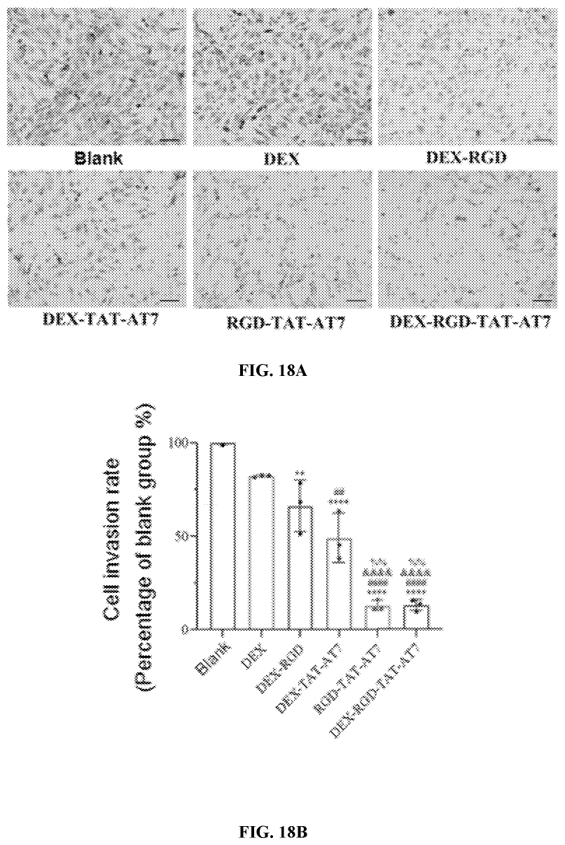

The results of the effect of DEX-RGD-TAT-AT7 on cell invasion are shown in FIGS. 18A and 18B. Compared to the control group, the difference in cell invasion rate in the DEX group has no significant meanings, while the differences in the other experimental groups compared to the control group have significant meanings. Where the cell invasion rates of RGD-TAT-AT7 and DEX-RGD-TAT-AT7 are only 13.06% and 13.28%, respectively, significantly lower than those of the other groups. The above results indicate that, compared to DEX-RGD and DEX-TAT-AT7 nanomicelles, DEX-RGD-TAT-AT7 has the best activity in inhibiting HUVECs cell invasion, and its inhibitory ability is comparable to that of RGD-TAT-AT7.

(3) The effect of DEX-RGD-TAT-AT7 on Tube Formation

One day in advance, 200 IL yellow pipette tips and a 96-hole plate are placed at −20° C. to pre-cool, and the matrix gel is placed at 4° C. to melt. The process of laying the matrix gel is performed on ice in the ultra-clean bench. 50 μL of the matrix gel pre-melted at 4° C. is added to each hole of the 96-hole plate and gently mixed well, and a sterile syringe needle is used to break the bubbles on the matrix gel. Then the 96-hole plate is incubated in a 37° C. incubator for 2 hours, waiting for the matrix gel to solidify. After digesting the logarithmic phase HUVECs, the logarithmic phase HUVECs are resuspended to adjust a cell concentration to be 1×10⁵ cells/mL. Then the 96-hole plate is removed from the incubator, the unsolidified matrix gel is aspirated using pipette tip, and 50 μL of the cell suspension is added to each hole. At the same time, 150 μL of the culture medium containing the conjugate nanomicelles and 1 μL of VEGF A165 protein are added, grouping as DEX group, DEX-RGD group, DEX-TAT-AT7 group, RGD-TAT-AT7 group, and DEX-RGD-TAT-AT7 group, and making the final concentration of the nanomicelles 40 μmol/L. Ten a control group is set up, with 50 μL of cell suspension, 150 μL of F12K basic culture medium, and 1 μL of VEGF A165 protein in each hole. Then the groups are incubated in the 37° C. incubator for 4 hours. After incubating, the 96-hole plate is taken out, photos are taken by an inverted fluorescence microscope using bright field mode, and Image J is used to calculate the number of newly formed tubular structures.

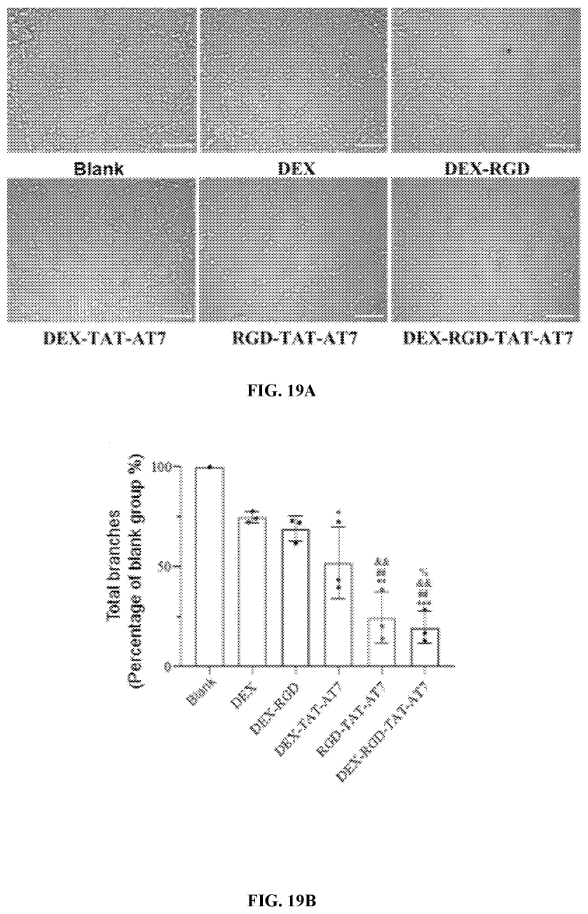

The results of the effect of DEX-RGD-TAT-AT7 on tube formation are shown in FIGS. 19A and 19B. Compared to the control group, the total branch length of blood vessels in the DEX group and that in the DEX-RGD group are slightly reduced, while the total branch length of blood vessels in the DEX-RGD-TAT-AT7 group is the lowest, and the differences with the other drug-administration groups have significant meanings. The above results indicate that DEX-RGD-TAT-AT7 can significantly inhibit the angiogenesis of HUVECs.

### Embodiment 9: In Vitro Anti-Inflammatory Activity Experiment

### (1) Establishment of Cell Inflammation Model

BV2 cells are cultured to the logarithmic phase, the cell density is adjusted to be 6×10⁵ cells/mL using DMEM complete culture medium, and the BV2 cells are plated in a 6-hole plate and incubated at 37° C. for 24 hours. After incubating, the original culture medium is aspirated, and LPS solution prepared with DMEM basic culture medium is added to each hole to induce inflammation in the cells, while also adding the culture medium containing the conjugate nanomicelles, making the final concentration of the nanomicelles 40 μmol/L and the final concentration of LPS 1 μg/mL. Then they are divided into blank group. LPS group, DEX group, RGD-TAT-AT7 group. DEX-RGD group. DEX-TAT-AT7 group, and DEX-RGD-TAT-AT7 group. The LPS group is a group without drug-containing culture medium, only LPS is given, and three sub-holes are set up, and is continued to culture for 24 hours. After the culture is completed, the cell supernatant is taken out for further ELISA inflammatory factor detection and NO detection.

### (2) ELISA Detection of Release of Inflammatory Factor

In step 1), the cell culture medium is collected, centrifuged at 1000×g for 20 minutes at 4° C. to remove impurities and cell debris, and the supernatant is taken for detection.

In step 2), the reagent kit is taken out of the refrigerator 20 minutes in advance and the reagent kit is equilibrated to room temperature.

In step 3). Preparation of washing solution: the concentrated washing solution is diluted with double distilled water at a ratio of concentrated washing solution:double distilled water=1:24.

In step 4). Preparation of standard working solution: the standard is centrifuged at 10000×g for 1 minute, then 1 mL of standard & sample diluent is added to the lyophilized standard. Then the tube cap is tightened and the tube is stood for 10 minutes. Then the tube is inverted for several times until fully dissolved, and the mixture is gently mixed well to avoid foaming. And then the standard is diluted using a serial dilution method to prepare the standard to be following concentrations: IL-6: 2000 pg/mL, 1000 pg/mL, 500 pg/mL, 250 pg/mL, 125 pg/mL, 62.5 pg/mL, 31.25 pg/mL, and 0 pg/mL, respectively; TNF-α: 500 pg/mL, 250 pg/mL, 125 pg/mL, 62.5 pg/mL, 31.25 pg/mL, 15.63 pg/mL, 7.81 pg/mL, and 0 pg/mL, respectively; iNOS: 20 ng/mL, 10 ng/mL, 5 ng/mL, 2.5 ng/mL, 1.25 ng/mL, 0.63 ng/mL, 0.31 ng/mL, and 0 ng/mL.

In step 5), standard holes, blank holes, and sample holes are set up respectively, 100 μL of the serial-diluted standard is added to the standard holes, 100 μL of standard & sample diluent is added to the blank holes, and 100 μL of samples to be tested is added to the remaining holes. When adding samples, the samples are added to the bottom of the ELISA plate, avoiding contact with walls of the hole. Then the ELISA plate is gently shaken to mix well to avoid generating bubbles. Finally, the ELISA plate is covered with membrane and incubated at 37° C. for 90 min.

In step 6), biotinylated antibody working solution/HRP enzyme conjugate working solution is prepared: Before the experiment, the amount needed for the experiment is calculated (calculated as 100 μL/hole), and 100~200 μL more than needed should be prepared. 15 minutes before use, the concentrated solution is centrifuged at 800×g for 1 minute, then the 100×concentrated solution is diluted to 1×working concentration with diluent (for example: 10 μL concentrated solution+990 μL diluent). The solution is prepared when the solution is needed to be used.

In step 7), the liquid in the holes is exhausted, and the holes are not needed to be washed. 100 μL of biotinylated antibody working solution is added to each hole, the ELISA plate is covered with a membrane and incubated at 37° C. for 1 hour.

In step 8), the liquid in the holes is exhausted and the ELISA plate is patted dry on clean absorbent paper. 350 μL of washing solution is added to each hole to soak each hole for 1 minute. Then the liquid in the ELISA plate is suctioned or discarded, and the ELISA plate is patted dry. These washing steps are repeated for 3 times.

In step 9), 100 μL of HRP enzyme conjugate working solution is added to each hole, then the ELISA plate is covered with a membrane and incubated at 37° C. for 30 minutes.

In step 10), the liquid in the holes is exhausted, and the plate is washed for 5 times, following the same method as that in step (8).

In step 11), 90 μL of substrate solution (TMB) is added to each hole, and the ELISA plate is covered with a membrane and incubated at 37° C. in the dark for 15 minutes.

In step 12), 50 μL of stop solution is added to each hole to stop the reaction. The order of adding the stop solution should be as consistent as possible with the order of adding the substrate solution.

In step 13), the absorbance (OD value) of each hole at a wavelength of 450 nm is immediately measured using the microplate reader. The concentrations of TNF-α, IL-6, and INOS in the samples are calculated according to the standard curves.

### (3) Detection of NO Concentration

1) Griess Reagent I and II are taken out, and allowed to return to room temperature.

2) The standard is diluted with DMEM basic culture medium to prepare the following concentrations: 0 μmol/L, 1 μmol/L, 2 μmol/L, 5 μmol/L, 10 μmol/L, 20 μmol/L, 40 μmol/L, 60 μmol/L, and 100 μmol/L.

3) 50 μL/hole of standard and sample are added to a 96-hole plate

4) 50 μL/hole of room temperature Griess Reagent I is added to each hole.

5) 50 μL/hole of room temperature Griess Reagent II is added to each hole.

6) The absorbance (OD value) at a wavelength of 540 nm is measured using the microplate reader.

7) The concentration of nitric oxide in the sample is calculated according to the standard curve.

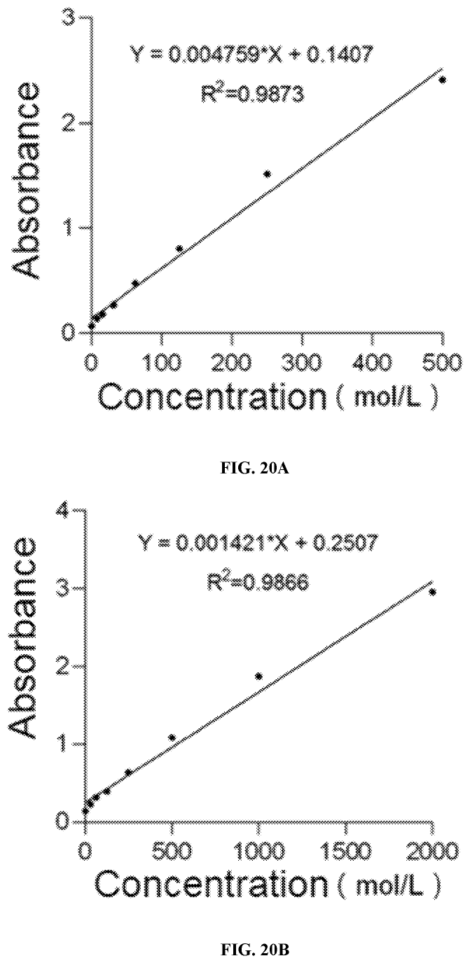

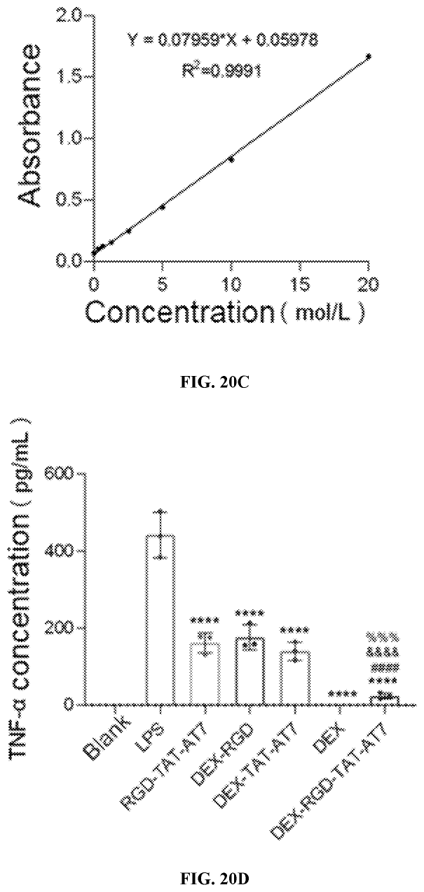

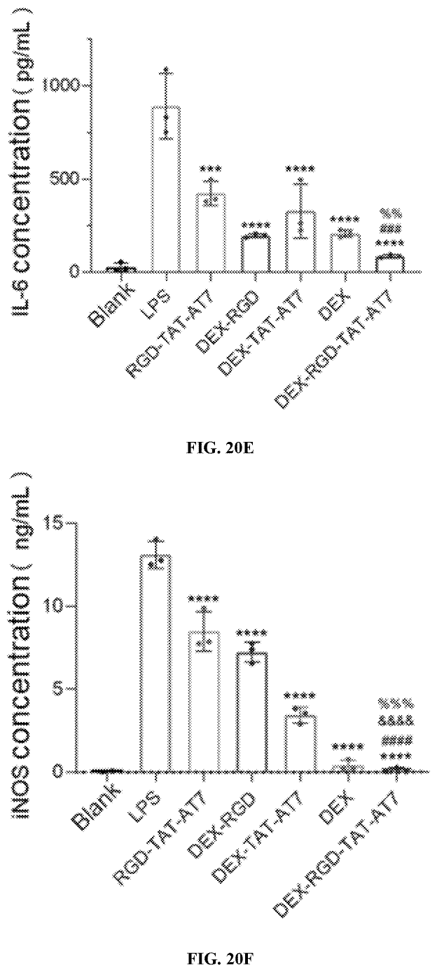

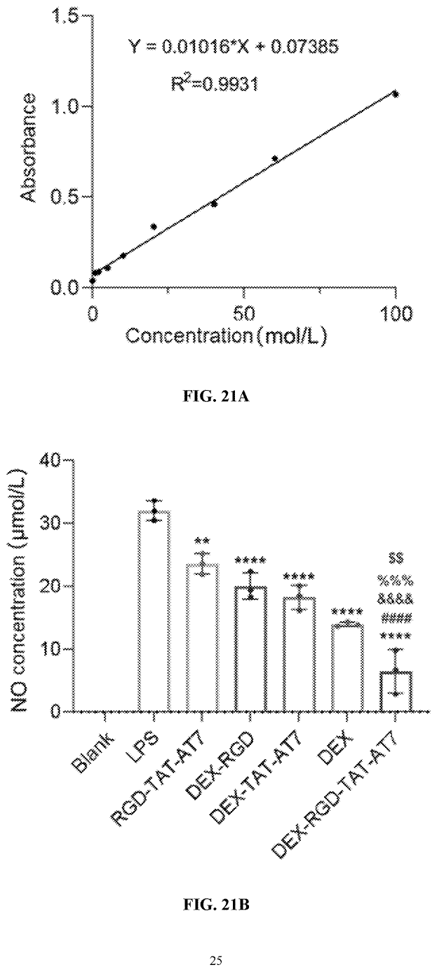

The results of the effect of DEX-RGD-TAT-AT7 on the release of inflammatory factors are shown in FIGS. 20A, 20B, 20C, 20D, 20E, 20F, 21A and 21B. In the determination of content of TNF-α, IL-6, and iNOS, the blank control group has almost no release of inflammatory factors, while after LPS stimulation, the release of inflammatory factors significantly increases, indicating that the inflammatory model stimulated by LPS is successfully constructed. The release of inflammatory factors in the drug administration groups is reduced compared to the LPS group, with the DEX group as the positive control group showing a more significant trend in inhibiting the release of inflammatory factors. Moreover, DEX-RGD-TAT-AT7 reduces the concentrations of TNF-α, IL-6, and iNOS compared to the LPS stimulation group by 94.6%, 92.46%, and 99.15%, respectively, and the differences between the DEX-RGD-TAT-AT7 group and the positive control DEX group have no significant meanings. In the measurement of NO release, the DEX-RGD-TAT-AT7 group shows a 79.85% decrease in NO release compared to the LPS stimulation group, outperforming other drug administration groups and the DEX group. The above results indicate that DEX-RGD-TAT-AT7 can inhibit the release of inflammatory factors from BV2 cells stimulated by LPS in vitro, and has a more significant activity in inhibiting the release of cell inflammatory factors compared to DEX-RGD, DEX-TAT-AT7, and RGD-TAT-AT7, while retaining similar anti-inflammatory activity to DEX.

### Embodiment 10: Hemolytic Investigation of DEX-RGD-TAT-AT7

1 mL of fresh rabbit blood is taken and added to an EP tube containing glass beads, and the EP tube is shaken for 10 minutes. 10 mL of physiological saline (0.9% NaCl solution) is added and mixed well. Then the mixture is centrifuged at 2000 r/min for 10 minutes, and then the supernatant is discarded, and the red blood cell precipitate is retained. Physiological saline is then added to wash for 2-3 times until the supernatant is no longer red. The obtained red blood cell precipitate is then resuspended using 50 mL of physiological saline and mixed well to prepare a 2% red blood cell suspension.

Three types of peptide-dexamethasone conjugate nanomicelles are prepared into physiological saline solutions with concentration gradients of 1000 μg/mL, 500 μg/mL, 250 μg/mL, 100 μg/mL, 50 μg/mL, 25 μg/mL, 10 μg/mL, 5 μg/mL, and 2.5 μg/mL, respectively. Then the double-distilled water is used as the positive control group and the physiological saline is used as the negative control group, 1.5 mL EP tubes are taken, 500 μL of the prepared drug solution and 500 μL of 4% red blood cell suspension are added to each tube and mixed well. The EP tubes are placed in a 37° C. thermostat water bath for incubating for 1 hour, then the hemolysis phenomenon is observed. 100 μL of the supernatant from each tube is taken into a 96-hole plate, and the absorbance (OD value) at a wavelength of 540 nm is measured using a microplate reader. The hemolysis rate is calculated using the following formula:

Hemolysis rate=(OD of drug administration group−OD of negative control group)/(OD of drug administration group−OD of positive control group)×100%

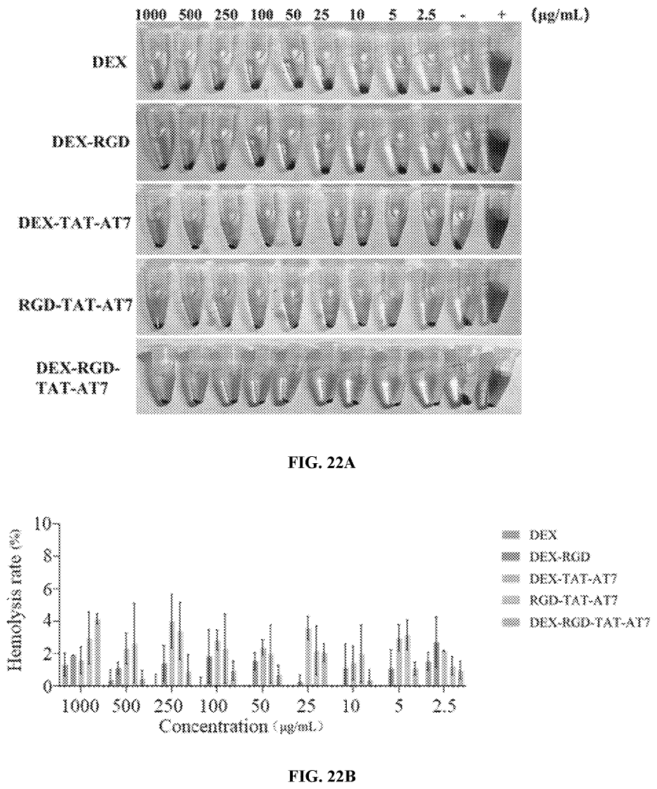

The results of the hemolytic investigation of DEX-RGD-TAT-AT7 are shown in FIGS. 22A and 22B. DEX-TAT-AT7, RGD-TAT-AT7, and DEX-RGD-TAT-AT7 between 100~1000 μg/mL exhibit slight hemolysis, but the hemolysis rates are all below 5%. The above results indicate that DEX-RGD, DEX-TAT-AT7, RGD-TAT-AT7, and DEX-RGD-TAT-AT7 have weak hemolytic properties and good in in vivo safety, and can be further used in in vivo experiments.

### Embodiment 11: In Vivo Anti-Inflammatory Activity of DEX-RGD-TAT-AT7

48 Kunming mice aged 6-8 weeks are prepared, with a male-to-female ratio of 1:1, and are randomly divided into 6 groups, with 8 mice in each group. Each group is continuously administered physiological saline, DEX, DEX-RGD, DEX-TAT-AT7. RGD-TAT-AT7, and DEX-RGD-TAT-AT7 by gavage for 7 days. One hour after the last drug administration on the 7th day, 20 μL of TPA dissolved in acetone is applied to the right ear of each mouse to induce inflammation, while the left ear is treated with acetone as a control After 4 hours, the mice are sacrificed, and both ears are cut off along the ear margin. A punch is used, and circular discs with a diameter of 8 mm are punched from the same position on both left and right ears and the left and right ears are weighed. The swelling degree is calculated using the following formula:

Swelling degree (mg)=Weight of right ear−Weight of left ear.

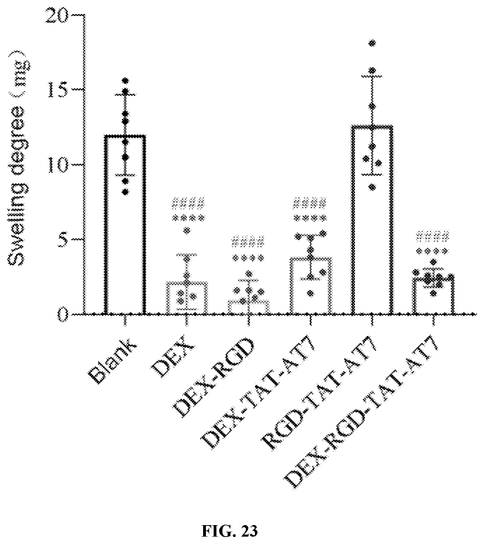

The results of the in vivo anti-inflammatory activity of DEX-RGD-TAT-AT7 are shown in FIG. 23. Compared to the average swelling degree of the control group (11.99 mg), the ear swelling degree of mice in the RGD-TAT-AT7 group is not decreased, while the ear swelling degrees of mice in the DEX-RGD group, DEX-TAT-AT7 group, and DEX-RGD-TAT-AT7 group decrease by 92.18%, 68.2%, and 79.67% compared to the control group, respectively, approaching 81.96% of the DEX group This indicates that the peptide-dexamethasone conjugate micelles can exert anti-inflammatory effects similar to dexamethasone, with DEX-RGD and DEX-RGD-TAT-AT7 showing the most significant inhibition of ear swelling in mice.

### Embodiment 12: In Vivo Anti-Angiogenic Eye Disease Activity of DEX-RGD-TAT-AT7

### (1) Construction of a Choroidal Neovascularization Model in Rats

24 brown Norway rats weighing 180~200 g are prepared and randomly divided into 8 groups. The rats are anesthetized using 2 mL of Avertin injection via intraperitoneal injection, and one drop of compound tropicamide eye drops is simultaneously dropped into the right eyes of the rats to dilate the pupils, and the whiskers on both sides of the rats are trimmed. After observing that the right eye of the rat is fully dilated under strong light, the rat is placed at the chin support of the laser treatment device, ensuring that the right eye's pupil is aligned with the laser emission port. A cover slip is taken and placed against the corneal of surface of the eye, and a krypton laser emitter is used to photocoagulate 8 points around the optic disc at the fundus. The power of the krypton laser emitter is set to 200 mW, with an exposure time of 50 ms and a spot diameter of 80 μm. The Bruch membrane is focused on, where bubbles may appear or slight bleeding may occur, indicating that the Bruch membrane has been ruptured, thereby indicating that the point is an effective photocoagulation point. No intervention is performed on the left eye.

### (2) Ocular Penetration of DEX-RGD-TAT-AT7

After establishing the CNV model. FITC-labeled nanomicelles are drug administered, and randomly grouped for drug administration as follows:
1) Negative control group: Physiological saline, eye drop administration, 20 μL, 6 times/day, for 2 days;
2) Positive control group: Conjunctival injection of FITC-RGD-TAT-AT7, 2 μL, single drug administration;
3) FITC-DEX-RGD: Eye drop administration, 20 μL, 6 times/day, for 2 days;
4) FITC-DEX-TAT-AT7: Eye drop administration, 20 μL, 6 times/day, for 2 days; and
5) FITC-DEX-RGD-TAT-AT7: Eye drop administration, 20 μL, 6 times/day, for 2 days.

After the last drug administration, the right eyeball of the rat is immediately removed and immersed in an EP tube containing eyeball fixation solution to fix at room temperature for 24 hours A sucrose solution is used for sugar precipitation treatment of the eyeball First, the eyeball is placed in 15% sucrose solution, then transferred to 30% sucrose solution, and dehydrated at 4° C. until the eyeball is sunk to the bottom of the EP tube. The eyeball is then rinsed with PBS twice, and the rinsed eyeball is transferred to a slicing box and placed on ice. Embedding agent OCT is dropped around the eyeball, and after the OCT turns white and hardens, a freezing microtome is used to make frozen sections with a thickness of 8 μm. The microscope slides with sections are placed into PBS to wash away the OCT embedding agent, and then the microscope slides with sections are fixed with 4% paraformaldehyde at room temperature for 25 minutes, and the sections are then washed with PBS for 3 times DAPI staining solution is dropped to the sections to stain in the dark at room temperature for 10 minutes. Then the sections are washed with PBS for 3 times, each time for 10 minutes. Then anti fluorescence quenching sealing agent is dropped on the microscope slides, and a cover slip is covered on the microscope slide for seal. A laser confocal microscope is used to observe the fluorescence intensity of the sections and quantitative analysis of the fluorescence is performed using Image J software.

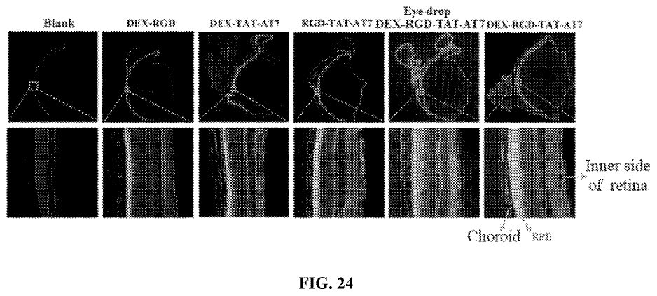

The results of the ocular penetration of DEX-RGD-TAT-AT7 are shown in FIG. 24. DEX-RGD, DEX-TAT-AT7, RGD-TAT-AT7, and DEX-RGD-TAT-AT7 all exhibit certain ocular penetration, able to penetrate from the anterior segment of the eye to the posterior segment, and after delivery to the choroid and retina outer layer, continue to penetrate the RPE layer to reach the inner layer of the retina. Compared to the DEX-RGD group, DEX-TAT-AT7 group, and RGD-TAT-AT7 group, the eye drop DEX-RGD-TAT-AT7 group shows the strongest fluorescence in the posterior segment of the eye, indicating the highest amount of drug reaching the posterior segment of the eye; the conjunctival injected DEX-RGD-TAT-AT7 group also exhibits strong fluorescence in the posterior segment of the eye. These results indicate that DEX-RGD-TAT-AT7 has certain ocular penetration, and the penetration effect of eye drop administration is comparable to that of direct conjunctival injection, allowing for further pharmacodynamic investigation.

### (3) Anti-Ocular Angiogenesis Activity of DEX-RGD-TAT-AT7

After establishing the CNV model, continuous drug administration for 14 days begins the next day, randomly grouped for drug administration as follows:
1) Negative control group: Physiological saline, Eye drop administration, 20 μL, 6 times/day, for 14 days;
2) Positive control group: Vitreous injection, Bevacizumab, 2 μL, 7 days/time;
3) DEX-RGD: Eye drop administration, 20 μL, 6 times/day, for 14 days;
4) DEX-TAT-AT7: Eye drop administration, 20 μL, 6 times/day, for 14 days;
5) RGD-TAT-AT7: Eye drop administration, 20 μL, 6 times/day, for 14 days;
6) DEX-RGD-TAT-AT7: Eye drop administration, 20 μL, 6 times/day, for 14 days; and
7) DEX-RGD-TAT-AT7: Subconjunctival injection, 2 μL, 7 days/time.

On the 7th and 14th days of drug administration, optical coherence tomography (OCT) is performed on the rat's eyeballs. The specific operations are as follows. The rat is anesthetized by intramuscular injection of 0.2 mL of Shutai. At the same time, compound tobramycin eye drops are applied to the left and right eyes to dilate the pupils. After the dilation is completed, sodium hyaluronate eye drops are applied to moisten the cornea, and the eyeballs are rubbed to remove excess reagents. The rat's pupils are aligned with the OCT lens, and OCT software is used to perform linear scans of the CNV lesion in the right eye and the normal retina in the left eye, calculating the CNV thickness.

After the last drug administration, the rats are euthanized, and choroidal flat mounts are prepared. First, the left and right eyeballs of the rat are removed with tweezers, carrying about 5 mm of the optic nerve, and soaked in eyeball fixation solution at room temperature for 20-30 minutes. The eyeballs are rinsed twice with PBS and soaked in a dish containing PBS, then transferred to operate under a body microscope. Ophthalmic scissors and ophthalmic forceps are used to carefully cut away the connective tissue and the optic nerve around the eyeballs An ophthalmic scissor is used to cut around the posterior ciliary body, and the lens along with the cornea are pushed out with ophthalmic forceps. The remaining posterior half of the eye cup's choroidal upper retinal portion is carefully removed with ophthalmic forceps, obtaining the choroid-sclera tissue complex, which is cut radially around the center of the optic disc with ophthalmic scissors to unfold it into a petal shape. The removed choroid-sclera tissue is transferred to a 5% BSA blocking solution and blocked at 4° C. for 24 hours. The choroid is rinsed with PBS, oscillating for 10 minutes, and repeated for three times. The fluorescein-labeled isolectin B4 (FITC-conjugated isolectin B4) is diluted to 20 μg/mL with PBS, and the choroid-sclera tissue is transferred to the staining solution, staining at room temperature for 24 hours. The choroid is then rinsed for three times with PBS as before, 10 μL of anti-fluorescence quencher is added to a clean microscope slide, excess moisture on the choroid-sclera tissue is absorbed, and the choroid-sclera tissue is transferred to the microscope slide, covered with a cover slip, and sealed with an anti-fluorescence quenching machine to minimize bubble formation in the sections. The sections can be stored at −20° C. for one month Fluorescence scanning of the sections is performed using a panoramic scanning microscope, and the CNV area is calculated using Image J software.

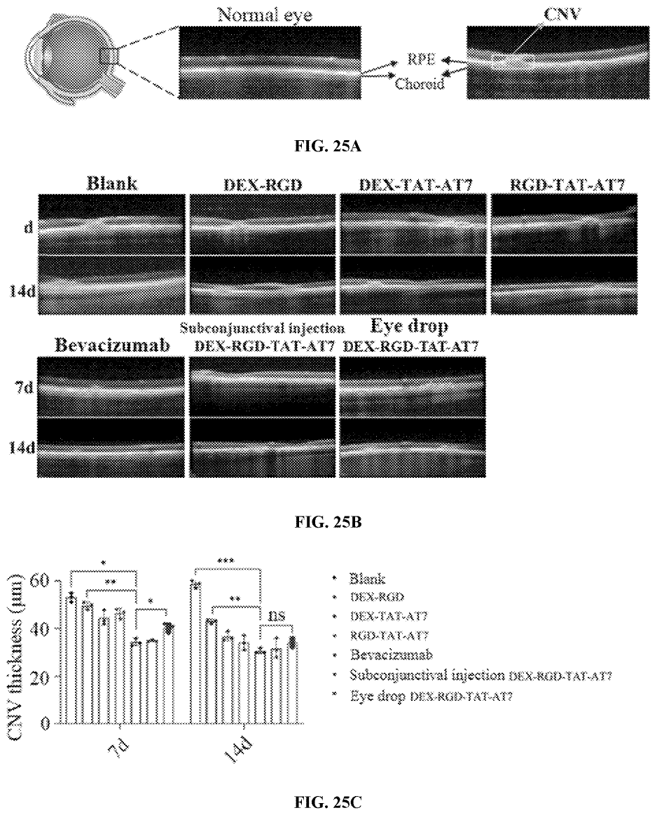

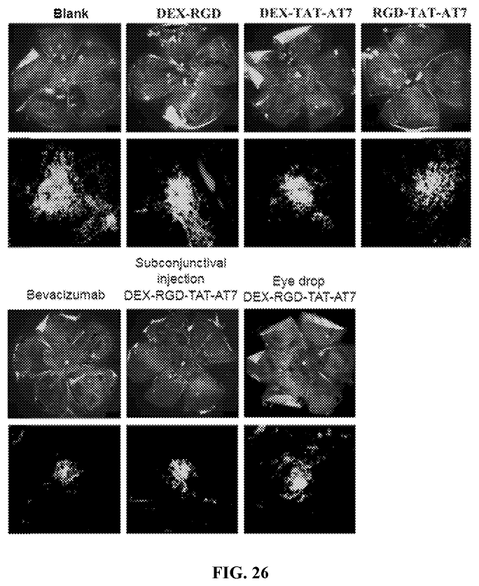

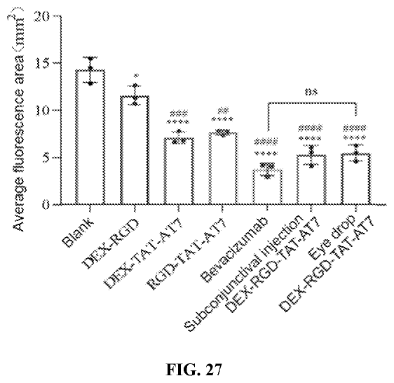

The anti-ocular angiogenesis activity results of DEX-RGD-TAT-AT7 are shown in FIGS. 25A, 25B and 25C, and FIGS. 26 and 27. After 14 days of drug administration, the CNV lesions in each drug-administrated group showed slight improvement compared to the CNV lesions at 7 days of drug administration, among which the CNV thickness in the eye drop administration DEX-RGD-TAT-AT7 group and that in the subconjunctival injection DEX-RGD-TAT-AT7 group are significantly reduced compared to the control group and the DEX-RGD group. And difference between the eye drop administration DEX-RGD-TAT-AT7 group and the bevacizumab group has no significant meanings. In the statistics of neovascular area, all drug-administrated groups show a reduction in neovascular area compared to the control group, among which the eye drop administration DEX-RGD-TAT-AT7 group and the subconjunctival injection DEX-RGD-TAT-AT group significantly improve CNV leakage compared to other groups, with the CNV area reduced by 61.71% and 63.23% respectively compared to the control group of 14.13 mm, and the difference in improvement effect among the eye drop administration DEX-RGD-TAT-AT7 group and the subconjunctival injection DEX-RGD-TAT-AT group as well as bevacizumab has no significant meanings. The above results indicate that eye drop administration of DEX-RGD-TAT-AT7 can improve the thickness of CNV lesions and the area of neovascularization in the choroid, showing a good anti-angiogenic effect on CNV, and is almost equivalent in efficacy to the subconjunctival injection group.

The above are only preferred embodiments of the present disclosure and are not intended to limit the present disclosure. For those skilled in the art, the present disclosure can have various modifications and changes. Any modifications, equivalent substitutions, improvements, etc., made within the spirit and principles of the present disclosure should be included within the protection scope of the present disclosure.

What is claimed is:

1. A peptide-dexamethasone conjugate, which chemical structure is selected from any of following chemical structures:

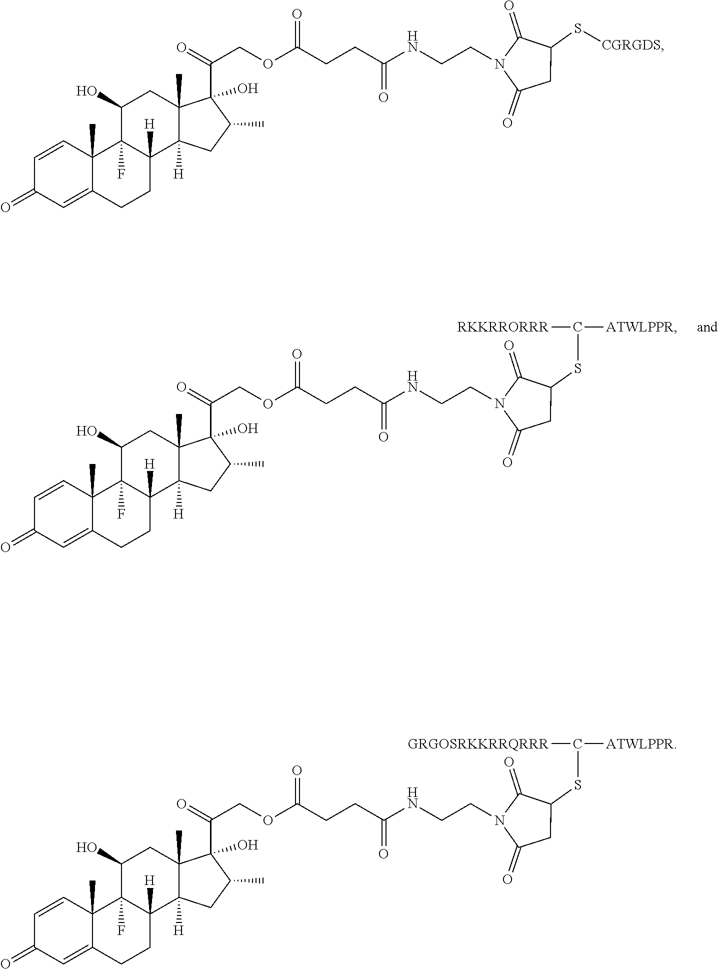

2. A preparation method of the peptide-dexamethasone conjugate according to claim 1, comprising: esterifying dexamethasone with succinic anhydride to obtain DEX-SA, then performing an amide condensation reaction of the DEX-SA with N-(2-aminoethyl) maleimide to obtain DEX-MAL, and finally conducting an addition reaction of the DEX-MAL with POI-SH to obtain the peptide-dexamethasone conjugate;
wherein a chemical structure of the DEX-SA is expressed as: 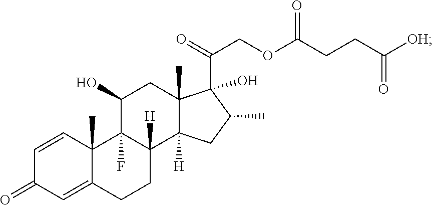
a chemical structure of the DEX-MAL is expressed as: 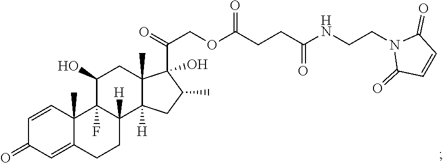
and the POI-SH is selected from HS-C-GRGDS,

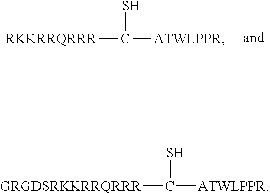

3. The preparation method of the peptide-dexamethasone conjugate according to claim 2, wherein dexamethasone with succinic anhydride and 4-dimethylaminopyridine are reacted at room temperature to obtain the DEX-SA.

4. The preparation method of the peptide-dexamethasone conjugate according to claim 2, wherein carboxylic acid in the DEX-SA is activated using 2-(7-azabenzotriazol-1-yl)-N,N,N′,N′-tetramethylurea hexafluorophosphate, 1-hydroxybenzotriazole, and N,N-diisopropylethylamine, and then N-(2-aminoethyl) maleimide is added to perform an amide condensation reaction to obtain the DEX-MAL.

5. The preparation method of the peptide-dexamethasone conjugate according to claim 2, wherein a condition for the addition reaction of the DEX-MAL with the POI-SH is: reacted at room temperature for 1.5~3 hours.

6. A pharmaceutical composition, comprising the peptide-dexamethasone conjugate according to claim 1, as well as pharmaceutical excipients.

7. A pharmaceutical formulation, comprising nanomicelles, which are formed by the peptide-dexamethasone conjugate according to claim 1.

8. Use of the peptide-dexamethasone conjugate according to claim 1 comprising formulating the peptide-dexamethasone conjugate in a preparation of a drug for treating neovascular eye diseases.

9. Use of the pharmaceutical composition according to claim 6 comprising formulating the peptide-dexamethasone conjugate in a preparation of a drug for treating neovascular eye diseases.

10. Use of the pharmaceutical formulation according to claim 7 comprising formulating the peptide-dexamethasone conjugate in a preparation of a drug for treating neovascular eye diseases.


SEQUENCE LISTING (5 sequences)

SEQ ID NO: 1 - protein, 5 aa, synthetic construct
GRGDS

SEQ ID NO: 2 - protein, 9 aa, synthetic construct
RKKRRQRRR

SEQ ID NO: 3 - protein, 7 aa, synthetic construct
ATWLPPR

SEQ ID NO: 4 - protein, 22 aa, synthetic construct
GRGDSRKKRRQRRRCATWLPPR

SEQ ID NO: 5 - protein, 17 aa, synthetic construct
RKKRRQRRRCATWLPPR